SEQUENCE LISTING (24 sequences)

SEQ ID NO: 1 - DNA, 1944 bp, Kaposi′s sarcoma-associated herpesvirus
ggatcccggcgcgccaccctccccggcaacaacctgttgccatgtatggcgatttgtatcagtcacaagcacacaacccctgctagtattaatggtgtttaaaacgttctacacgtacggcggaccgcatccgtcgcaagcacgcgcatataacccccaaatgcaccatgatgagaagcacagccacgcgtcaaaaaactttaaaaacatcgttatccaatatcattaaaaaccacaccgaaatttacacaggtagcacgtcaccgtgttagtgtcacccactgtacacaaggcgtgtcgtatatgtagtataggtatttgatgaggcggaagcatatcccgcttccagcgaacggaaataagaatcatccgttccagcatttattcaaagagggcacagaggattcacattgtttagagagagtttttcttagtcaccattccatacttgggcagtattggcctacgatttgggcgacgtttcaggctggtctattctccgtccacttttccccggctattctgtcccagcataggctcttgaaataaacaatgtttaccgagtaaaaggttccactcaccctcatttgtcgttgcacccatcccccctttgcttaatcacccgaaaactagaggacacggatggaaaacatatcgcacgcgggttgtttgaaagtcaacagctacttgtttttaatgaggacagatttgggcacaggccagagggtaaagccctacgtgtgcgcgggggggggggtgtatacgctgcgaaaacctgcacggtgcataacacccagggcgtcacgtcacatatctctgtgcacccaagtggttgttcaaccgttgttttttggatgatttttccgcaccggcttttttgtgggcgcgcataggtcggtacgcgctgtccccctaagtcccgcacggtcgttcgggcccccgtccggctcgtctccggatgaaccgtcacgttctttgtctccagaggcgacgtctccttcagatgactcgtccgtgggctcctcgtccgtcccgcccgcgggtccgacaaggaccgtcaattcgatgttatcttcgttcgcggttggccggcgcggccgtcggtatggcagtacggtcacccgggtgttatttgccgcgtataatgccctcacagtgccacttacgcggcatatgccgccaaatgcaaacacaataaatatttggtaaaacccaaagaagcagagaaaaccgagcacggccccgggggagaatgttcccgcaggagcagttaggatgaccaggagcgtccaggtgcacaacgccacgccgacaagcccagccaccaccacagacatcagcagaaacagttcaaaaatttcttggcgctccatctccggccacaggttaaggcgactacgccactgcgtgcgcgtgcggtatataacgcgacacatttgacaggccgtgtttcgagacactgttagccaagtgcttaaacactgcgggtggacgacatccagctctccggtacaggcgcaggggtgtatgccctcgttccccacctcttccctacatatccagcagatgggtccctctacaccctcttctacgtccttagacgccatctctgcagctggggtggaagtctgaaaaagggaaaggggaggtgagcagagtgcccagttagtctccgacccgccgtccgccctactgtcgctatcccgccttgacagatgtctaacgtattcacggacgccacatgtgtgtctattttcctacatccaggctttccctggaaaactgtcacaacccaccctgctttagctctacatctgtatttttgtttacgcacaggatcaacgcttcgtgcccgtccacccccgcgctctccgcctgtgtttggaggttttatgagtggttagttctaggcagctccggacaagttgtccaaaacacggcgcg

SEQ ID NO: 2 - DNA, 836 bp, Kaposi′s sarcoma-associated herpesvirus
ggatcccggcgcgccaccctccccggcaacaacctgttgccatgtatggcgatttgtatcagtcacaagcacacaacccctgctagtattaatggtgtttaaaacgttctacacgtacggcggaccgcatccgtcgcaagcacgcgcatataacccccaaatgcaccatgatgagaagcacagccacgcgtcaaaaaactttaaaaacatcgttatccaatatcattaaaaaccacaccgaaatttacacaggtagcacgtcaccgtgttagtgtcacccactgtacacaaggcgtgtcgtatatgtagtataggtatttgatgaggcggaagcatatcccgcttccagcgaacggaaataagaatcatccgttccagcatttattcaaagagggcacagaggattcacattgtttagagagagtttttcttagtcaccattccatacttgggcagtattggcctacgatttgggcgacgtttcaggctggtctattctccgtccacttttccccggctattctgtcccagcataggctcttgaaataaacaatgtttaccgagtaaaaggttccactcaccctcatttgtcgttgcacccatcccccctttgcttaatcacccgaaaactagaggacacggatggaaaacatatcgcacgcgggttgtttgaaagtcaacagctacttgtttttaatgaggacagatttgggcacaggccagagggtaaagccctacgtgtgcgcgggggggggggtgtatacgctgcgaaaacctgcacggtgcataacacccagggcgtcacgtcacatatctctgtgcacccaagtggttgt

SEQ ID NO: 3 - DNA, 771 bp, Kaposi′s sarcoma-associated herpesvirus
tcaaccgttgttttttggatgatttttccgcaccggcttttttgtgggcgcgcataggtcggtacgcgctgtccccctaagtcccgcacggtcgttcgggcccccgtccggctcgtctccggatgaaccgtcacgttctttgtctccagaggcgacgtctccttcagatgactcgtccgtgggctcctcgtccgtcccgcccgcgggtccgacaaggaccgtcaattcgatgttatcttcgttcgcggttggccggcgcggccgtcggtatggcagtacggtcacccgggtgttatttgccgcgtataatgccctcacagtgccacttacgcggcatatgccgccaaatgcaaacacaataaatatttggtaaaacccaaagaagcagagaaaaccgagcacggccccgggggagaatgttcccgcaggagcagttaggatgaccaggagcgtccaggtgcacaacgccacgccgacaagcccagccaccaccacagacatcagcagaaacagttcaaaaatttcttggcgctccatctccggccacaggttaaggcgactacgccactgcgtgcgcgtgcggtatataacgcgacacatttgacaggccgtgtttcgagacactgttagccaagtgcttaaacactgcgggtggacgacatccagctctccggtacaggcgcaggggtgtatgccctcgttccccacctcttccctacatatccagcagatgggtccctctacaccctcttctacgtccttagacgccat

SEQ ID NO: 4 - DNA, 11 bp, Saccharomyces cerevisiae
taaacataaaa

SEQ ID NO: 5 - DNA, 11 bp, Kaposi′s sarcoma-associated herpesvirus
aaaactttaaa

SEQ ID NO: 6 - DNA, 21 bp, Artificial
  primer
tgctagtattaatggtgttta

SEQ ID NO: 7 - DNA, 18 bp, Artificial
  primer
tgacgtgctacctgtgta

SEQ ID NO: 8 - DNA, 25 bp, Artificial
  primer
tggcgtctaaggacgtagaagaggg

SEQ ID NO: 9 - DNA, 20 bp, Artificial
  primer
tgcgtgcgcgtgcggtatat

SEQ ID NO: 10 - DNA, 21 bp, Artificial
  primer
tgctagtattaatggtgttta

SEQ ID NO: 11 - DNA, 18 bp, Artificial
  primer
tgacgtgctacctgtgta

SEQ ID NO: 12 - DNA, 22 bp, Artificial
  primer
atccatgccgcgagggtatagg

SEQ ID NO: 13 - DNA, 20 bp, Artificial
  primer
tatgttggtgcccagtcggg

SEQ ID NO: 14 - DNA, 18 bp, Artificial
  primer
agcccccgatttagagct

SEQ ID NO: 15 - DNA, 18 bp, Artificial
  primer
cagcctgaatggcgaatg

SEQ ID NO: 16 - DNA, 1970 bp, Kaposi′s sarcoma-associated herpesvirus
ggagggggatcccggcgcgccaccctccccggcaacaacctgttgccatgtatggcgatttgtatcagtcacaagcacacaacccctgctagtattaatggtgtttaaaacgttctacacgtacggcggaccgcatccgtcgcaagcacgcgcatataacccccaaatgcaccatgatgagaagcacagccacgcgtcaaaaaactttaaaaacatcgttatccaatatcattaaaaaccacaccgaaatttacacaggtagcacgtcaccgtgttagtgtcacccactgtacacaaggcgtgtcgtatatgtagtataggtatttgatgaggcggaagcatatcccgcttccagcgaacggaaataagaatcatccgttccagcatttattcaaagagggcacagaggattcacattgtttagagagagtttttcttagtcaccattccatacttgggcagtattggcctacgatttgggcgacgtttcaggctggtctattctccgtccacttttccccggctattctgtcccagcataggctcttgaaataaacaatgtttaccgagtaaaaggttccactcaccctcatttgtcgttgcacccatcccccctttgcttaatcacccgaaaactagaggacacggatggaaaacatatcgcacgcgggttgtttgaaagtcaacagctacttgtttttaatgaggacagatttgggcacaggccagagggtaaagccctacgtgtgcgcgggggggggggtgtatacgctgcgaaaacctgcacggtgcataacacccagggcgtcacgtcacatatctctgtgcacccaagtggttgttcaaccgttgttttttggatgatttttccgcaccggcttttttgtgggcgcgcataggtcggtacgcgctgtccccctaagtcccgcacggtcgttcgggcccccgtccggctcgtctccggatgaaccgtcacgttctttgtctccagaggcgacgtctccttcagatgactcgtccgtgggctcctcgtccgtcccgcccgcgggtccgacaaggaccgtcaattcgatgttatcttcgttcgcggttggccggcgcggccgtcggtatggcagtacggtcacccgggtgttatttgccgcgtataatgccctcacagtgccacttacgcggcatatgccgccaaatgcaaacacaataaatatttggtaaaacccaaagaagcagagaaaaccgagcacggccccgggggagaatgttcccgcaggagcagttaggatgaccaggagcgtccaggtgcacaacgccacgccgacaagcccagccaccaccacagacatcagcagaaacagttcaaaaatttcttggcgctccatctccggccacaggttaaggcgactacgccactgcgtgcgcgtgcggtatataacgcgacacatttgacaggccgtgtttcgagacactgttagccaagtgcttaaacactgcgggtggacgacatccagctctccggtacaggcgcaggggtgtatgccctcgttccccacctcttccctacatatccagcagatgggtccctctacaccctcttctacgtccttagacgccatctctgcagctggggtggaagtctgaaaaagggaaaggggaggtgagcagagtgcccagttagtctccgacccgccgtccgccctactgtcgctatcccgccttgacagatgtctaacgtattcacggacgccacatgtgtgtctattttcctacatccaggctttccctggaaaactgtcacaacccaccctgctttagctctacatctgtatttttgtttacgcacaggatcaacgcttcgtgcccgtccacccccgcgctctccgcctgtgtttggaggttttatgagtggttagttctaggcagctccggacaagttgtccaaaacacggcgcgccccgcccttccttccctcc

SEQ ID NO: 17 - RNA, 19 nt, Artificial
  primer
auccugagauuacgauaaa

SEQ ID NO: 18 - RNA, 19 nt, Artificial
  primer
cuuacgcugaguacuucga

SEQ ID NO: 19 - DNA, 30 bp, Artificial
  primer
aatggatccagagacccatatgtgatcacg

SEQ ID NO: 20 - DNA, 27 bp, Artificial
  primer
aatggatcctcgactctgctccggtgg

SEQ ID NO: 21 - DNA, 20 bp, Artificial
  primer
ccgcgcagcaacagatggaa

SEQ ID NO: 22 - DNA, 20 bp, Artificial
  primer
aagccgagccgctcgtagaa

SEQ ID NO: 23 - DNA, 19 bp, Artificial
  primer
gccacatcgagcgggtcac

SEQ ID NO: 24 - DNA, 18 bp, Artificial
  primer
tcggcggtgacggtgaag

(19) United States
(12) Patent Application Publication
(10) Pub. No.: US 2009/0221468 A1
(43) Pub. Date: Sep. 3, 2009

(54) Autonomously Replicating KSHV CIS-Acting Elements

(76) Inventors: Erle S. Robertson, Wynnewood, PA (US); Subhash C. Verma, Philadelphia, PA (US)

Correspondence Address: Pearl Cohen Zedek Latzer, LLP, 1500 Broadway, 12th Floor, New York, NY, 10036, US

(21) Appl. No.: 12/083,730

(22) PCT Filed: Oct. 17, 2006
(86) PCT No.: PCT/US2006/040713

§ 371 Date: May 5, 2009

Related U.S. Application Data
(60) Provisional application No. 60/726,606, filed on Oct. 17, 2005.

(51) Int. Cl. A61K 38/00; C12Q 1/68; C07H 21/04; A61K 31/7088

(52) U.S. Cl. 514/2; 435/6; 536/23.1; 514/44 R

(57) ABSTRACT

This invention provides methods of reducing a replication of a gammaherpesvirus genome, treating a KSHV infection, and treating or reducing an incidence of a KSHV-associated disease, comprising contacting a subject with a composition that inhibits initiation of DNA replication from a region of a genome of a gammaherpesvirus. The invention also provides isolated DNA molecules capable of episomal replication in a eukaryotic cell, and methods of delivering a recombinant protein or therapeutic RNA molecule, comprising same.

### FIELD OF INVENTION

This invention provides methods of reducing a replication of a gammaherpesvirus genome, treating a KSHV infection, and treating or reducing an incidence of a KSHV-associated disease, comprising contacting a subject with a composition that inhibits initiation of DNA replication from a region of a genome of a gammaherpesvirus. The invention also provides isolated DNA molecules capable of episomal replication in a eukaryotic cell, and methods of delivering a recombinant protein or therapeutic RNA molecule, comprising same.

### BACKGROUND OF THE INVENTION

KSHV is a member of the rhadinovirus genera of the gammaherpesvirinae subfamily, and is related to herpesvirus saimiri (HVS) and rhesus rhadinovirus (RRV). Epstein-Barr virus (EBV), also known as human herpesvirus 4 (HHV4), is the prototypic member of the other gammaherpesvirinae genera, lymphocryptovirus. KSHV is associated with several cancers and tumors, e.g. Kaposi's sarcoma (KS), primary effusion lymphoma (PEL), and multicentric Castleman's disease. KSHV infection within tumor cells is predominantly latent and involves the characteristic maintenance of viral genomes as circular DNA episomes.

The KSHV latency-associated nuclear antigen (LANA) has been shown to play roles in both transcription and plasmid maintenance. LANA is a large, 222-234 kDa nuclear protein that is expressed in virtually all cells latently infected with KSHV, and has been shown to protect against cell death by inhibition of p53-mediated transcription. Additionally, LANA tethers viral episomes to metaphase chromosomes and binds to host chromosomes, allowing for efficient persistence of KSHV episomal DNA. LANA-independent KSHV replication has not been demonstrated to date.

Protein origin replication complexes (ORC) bind to specific DNA sites on the genome, as the first step in assembly of the pre-RC (replication complex), determining the site of initiation of DNA replication. ORCs recruit the replicative helicase (MCM), followed by the initiation of DNA replication at each origin.

### SUMMARY OF THE INVENTION

This invention provides methods of reducing a replication of a gammaherpesvirus genome, treating a KSHV infection, and treating or reducing an incidence of a KSHV-associated disease, comprising contacting a subject with a composition that inhibits initiation of DNA replication from a region of a genome of a gammaherpesvirus. The invention also provides isolated DNA molecules capable of episomal replication in a eukaryotic cell, and methods of delivering arecombinant protein or therapeutic RNA molecule, comprising same.

In one embodiment, the present invention provides a method of reducing a replication of a genome of a gammaherpesvirus in a subject, comprising contacting the subject with a composition that inhibits initiation of DNA replication from a region of a genome of the gammaherpesvirus, thereby reducing a replication of a genome of a gammaherpesvirus in a subject. In another embodiment, the region has a sequence set forth in SEQ ID No: 2. In another embodiment, the sequence of the region is a fragment of SEQ ID No: 2. In another embodiment, the region is homologous to SEQ ID No: 2. In another embodiment, the region is homologous to a fragment of SEQ ID No: 2. In another embodiment, the sequence of the region comprises SEQ ID No: 5. In another embodiment, the sequence of the region consists of SEQ ID No: 5. Each possibility represents a separate embodiment of the present invention.

In another embodiment, the present invention provides a method of treating a KSHV infection in a subject, comprising contacting the subject with a composition of the present invention, thereby treating a KSHV infection in a subject.

In another embodiment, the present invention provides a method of treating or reducing an incidence of a KSHV-associated KS, PEL, or multicentric Castleman's disease in a subject, comprising contacting the subject with a composition of the present invention, thereby treating or reducing an incidence of a KSHV-associated KS, PEL, or multicentric Castleman's disease in a subject.

In another embodiment, the present invention provides an isolated DNA molecule, the isolated DNA molecule comprising (a) a non-Kaposi's Sarcoma-Associated Herpesvirus (KSHV) portion; and (b) a region of a KSHV genome. In another embodiment, the sequence of the region is set forth in SEQ ID No: 2. In another embodiment, the sequence of the region is a fragment of SEQ ID No: 2. In another embodiment, the region is homologous to SEQ ID No: 2. In another embodiment, the region is homologous to a fragment of SEQ ID No: 2. In another embodiment, the sequence of the region consists of SEQ ID No: 2. In another embodiment, the sequence of the region comprises SEQ ID No: 5. In another embodiment, the region has a sequence consisting of SEQ ID No: 5. In another embodiment, the isolated DNA molecule is capable of episomal replication in a eukaryotic cell. Each possibility represents a separate embodiment of the present invention.

In another embodiment, the present invention provides a method of delivering a recombinant protein to a subject, comprising administering to the subject a DNA molecule of the present invention, thereby delivering a recombinant protein to a subject. In another embodiment, the DNA molecule further comprises a recombinant gene. The recombinant gene, in another embodiment, encodes the recombinant protein to be delivered.

In another embodiment, the present invention provides a method of delivering a therapeutic RNA molecule to a subject, comprising administering to the subject a DNA molecule of the present invention, wherein the DNA molecule further comprises a recombinant gene, the recombinant gene encoding the therapeutic RNA molecule, thereby delivering a therapeutic RNA molecule to a subject.

### BRIEF DESCRIPTION OF THE FIGURES

FIG. 1. A. Evaluation of the replication potential of Z6 cosmid fragments. (A) Schematic of KSHV genome. Z6, left end of KSHV genome, digested with the indicated enzymes generating fragments A-H. These fragments were cloned into pBSpuro at respective enzyme sites. Clones containing single and 2 copies of terminal repeats were termed pBSpuroA and pBSpuroA3. pBSpuroB clone, corresponding to the extreme left end of the genome, had the K1 ORF as well as a region of the TR. The C fragment (14.8 kb), obtained by BamHI and NotI digestion, was further digested with XbaI, yielding three fragments pBSpuro (C×1, C×2 and C×3). Fragments D-H were also created and subcloned into pBS. (B) DpnI sensitivity assay of fragments A-H. Lane E, 10% of the total Hirt's DNA digested with EcoRI to linearize, Lane E+D, digested with EcoRI and DpnI. Arrow indicates the pBSpuro-spiked plasmid to test completion of digestion. Asterisks indicate the replicated copies of the plasmids. DpnI resistant band intensities were quantified using Kodak Gel Logic 1D image software normalizing input as 10 and the relative quantities of DpnI bands are shown in the bar diagram. (C) DpnI analysis of pBSpuroG and pBSpuro into human foreskin fibroblast (R2F), Rat-1 and DG-75. DpnI resistant band intensities were calculated based on input at 5% and the relative amounts of DpnI resistant bands are depicted as a bar diagram.

FIG. 2. pBSpuroG replicates and incorporates density label in a cell cycle manner similar to the cellular DNA. 293 cells transfected with either pBSpuroG or pBSpuro were labeled with 30 μg/ml BrdU for 0 h, 24 h or 48 h. DNA extracted from these cells was sheared and separated on CsCl gradient centrifugation. Distribution of total DNA from pBSpuroG-(A) and pBSpuro-(B) transfected 293 cells in all 16 fractions of CsCl density gradient after Oh; triangles, 24 h; squares and 48 h; circles, were quantified using Sybr Green and plotted against density of each fraction. Distribution of DNA was detected as 3 peaks at the densities 1.7, 1.75 and 1.8 g/cm³, which correspond to the un-replicated light:light (L:L), semi-replicated heavy:light (H:L) and fully replicated heavy:heavy (H:H) DNA molecules, respectively. Relative copies of the pBSpuroG (C) and pBSpuro (D) in each fraction, quantified in a semi-quantitative PCR amplifying puro gene as a target sequence, are shown as black bars. Gray bars: relative distribution of cellular DNA quantified based on the amplification of GAPDH gene.

FIG. 3, part 1. pBSpuroG persists as an episomal DNA detected by in-situ lysis. (A) Gardella gel analysis of the long term selected clones. Left panel: EtBr stained gel which was transferred onto the gene screen membrane and hybridized using ³²P-labeled puro gene as a probe, as depicted in the right panel, which shows the presence of different forms of the plasmid. Lanes 1, 2 and 3: pBSPuroG plasmid selected clones; lane 4: purified pBSpuroG (3 ng); lane 5: clones with pBSpuro plasmid; lane 6: purified pBSpuro (3 ng). Chr DNA: chromosomal DNA. Arrows indicate plasmids pBSpuroG and pBSpuro in respective lanes. Triangles indicate hybridization signal of chromosomal DNA in pBSpuro plasmid selected clone. (B) Localization of pBSpuroG in long term-selected colonies by fluorescence in-situ hybridization. Chromosomes spreads of pBSpuroG long term-selected cells were hybridized with biotin-labeled probe and detected with streptavidin conjugated with Alexa flour 594 (red). Red dots: hybridization signals indicating the presence of pBSpuroG. (C) Chromosome spreads of HEK293 cells were used as a control to exclude non-specific cross-hybridizing signals. (D) Average number of hybridizing dots per chromosome calculated based on 10 optical fields. Part 2. B/W version of B-C: red fluorescence alone is shown as white; blue fluorescence is shown as gray.

FIG. 4: Long-term selected pBSpuroG plasmid is replicated in its native form. (A) DpnI sensitivity assay of Hirt DNA extracted from 4 long term-selected clones. Lane E (EcoRI) and E+D (EcoRI and DpnI) pBS were added in each reaction to test for completion of digestion. (B) Number of colonies yielded after transformation of the DpnI digested Hirt DNA extracted from long term-selected pBSpuroG and pBSpuro plasmids. (C) Restriction patterns of isolated plasmid from transformants with BamHI (C) and PstI (D). Lane 1: pBSpuro; lane 2: pBSpuroG; lanes 3-10: plasmid DNA isolated from the colonies yielded with DpnI-digested long term selected Hirt DNA.

FIG. 5. AT Region can support replication. (A) Schematic of the G fragment DNA sequence between coordinates 24877-25713 nt of KSHV genome. (B) Hirt DNA extracted from 293 cells transfected with G, AT or K5 regions of G independently were digested with EcoRI (5%, to linearize) or EcoRI +DpnI, and DpnI-resistant bands were quantified. Intensities of input EcoRI lane was set to 5 and relative intensities of DpnI band in each lane were calculated based on the respective inputs. AT region-containing plasmid (pBSpuro gAT) and control AT region of KSHV genome-containing plasmids (pBSpuro cAT) were transfected into 293 cells for DpnI sensitivity assay. 10% of the total Hirt DNA was digested with EcoRI and the remainder with E+D. Relative numbers of DpnI-resistant copies were calculated, compared to the input copies (set at 10). (C) Thymidine growth-arrested PELs cells at G1/S phase were used for chromatin immunoprecipitation. (D) Western blots showing IP of chromatins using α-ORC2 and α-MCM3 antibodies (lane 3). Lane 2, control IgG showed lack of non-specific IP of chromatin. Lane 1; Input (10% total chromatin). Lane 4, proteins in post-ChIP. (E) Coordinates of KSHV genome indicating E, and G regions. (F) Quantitation of the relative number of E and AT genomic copies in the chromatins immunoprecipitated with α-ORC2 and α-MCM3 antibodies from PELs (BC-3, BCBL-1 and JSC-1). (G) Amplification of puro gene (adjacent to the G, AT or K5 region) in the DNA recovered from immunoprecipitated chromatins of 293 cells transfected with pBSpuroG, pBSpuroAT and pBSpuroK5 and vector control pBSpuro using α-ORC2 and α-MCM3 antibodies. Relative amounts of amplicon in chromatins were quantified based on the amplification of input lane as 10%. (H) Cellular ORC2 and MCM3 do not bind to control AT region of KSHV genome. Western blot shows IP of chromatin with α-ORC2 and α-MCM3 antibodies from pBSpuro gAT and pBSpuro cAT. Absence of amplicon in DNA from chromatins of pBSpuro cAT (lanes 7 and 8) compared to the pBSpuro gAT (lanes 3 and 4) indicates no binding of cellular replication proteins to chromatin.

FIG. 6. pBSpuroG actively incorporates BrdU and does not replicate in ORC2-depleted cells. (A) A schematic of BrdU incorporation and IP of BrdU labeled DNA. α-BrdU antibody immunoprecipitated BrdU incorporated newly synthesized DNA from TR with LANA expression as well as G fragment containing plasmid detected by the amplification of immunoprecipiated DNA in lanes 3. Lane 1: input; Lane 2: IP of BrdU-labeled DNA with control IgG antibody. Vector control (pBSpuro) did not exhibit detectable level of amplification. (B) G fragment-containing plasmid replicates in absence of LANA. DpnI sensitivity assay of Hirt DNA extracted from 293 cells transfected with pBSpuroTR and pBSpuroG with (+) and without (−) LANA-expressing cells. Quantitation of DpnI-resistant copies based on input, 10% in EcoRI lanes. (C) Western blots showing efficient depletion of ORC2 expression in ORC2 specific siRNA compared to the scrambled siRNA transfected 293 cells. (D) Evaluation of pBSpuroG mediated replication by BrdU incorporation in 293 cells depleted with ORC2. Anti-BrdU antibody failed to IP G fragment-containing plasmid from ORC2 siRNA treated cells (lane 3, ORC2 siRNA), whereas scrambled siRNA-treated cells exhibited IP of BrdU-labeled DNA (lane 3). Lane 4: control for amplification; lane 5: water as template (negative control).

FIG. 7: Single Molecule Analysis of the Replicated DNA. (A) Schematic showing incorporation of nucleotides in DNA element containing origin of replication (ori) site. (B) Images of 3 PmeI-linearized pBSpuroG DNA molecules (top panel). Hybridization signal detects pBSpuro backbone, shown in red. The G fragment, cloned into BamHI sites of pBSpuro, is flanked by regions of pBSpuro, which exhibit no hybridization signal. Incorporation of IdU was detected by mouse α-IdU antibody followed by Alexa Flour 350 (blue) showed labeling of entire length of the molecule after 1 round of replication with IdU. Pulsing with second label, CldU (green) for a short period (2 h) allowed determination of the transition site, which is the initiation site for the DNA replication. pBSpuro (bottom panel) did not exhibit incorporation of halogenated nucleotides due to the lack of replication initiation sites. (C) Model of KSHV persistence with a LANA-independent origin of replication. LANA tethers the viral genome to the host chromosome and persists as highly ordered chromatin structure. Binding of ORCs and MCMs at the G region of the KSHV allows binding of other proteins of replication machinery to make a pre-RC, thus acting as an origin of replication for latent replication of the KSHV genome. For A-C, rows are (top to bottom): probe, IdU; CldU; IdU+CldU merge; and merge (D). Detection of pBSpuroG molecules using probe directed against puromycin and ampicillin genes. Genes were excised from pBSpuro by restriction digest, followed by biotin labeling using random primer labeling kit (NEB). Purified probes were hybridized at 65° C. overnight under moist condition with the IdU and CldU labeled pBSpuroG or pBSpuro stretched on poly-L-lysine coated slides. Slides were washed, with 2×SSC+0.05% SDS and 0.1×SSC+0.05% SDS for 15 min each, post-hybridization. IdU and CldU in the labeled DNA were detected using mouse mAb α-IdU (Becton-Dickinson Inc., Palo Alto, Calif.) and Alexa Fluor 350 (blue) conjugated goat α-mouse (Molecular Probes) as primary and secondary antibodies. CldU was detected using rat anti-CldU (Accurate Chemicals, Westbury, N.Y.) and goat anti-rat conjugated with Alexa Flour 488 (green) secondary antibody. The G fragment lies in between the BamHI region which has an AccI site that divides the AT and K5 regions of the G fragment. Detection of transition site in AT region shows replication initiation site in this region of the G fragment. Merge panel shows that same molecule was visualized. pBSpuro molecules were also detected using the same probe and antibody. Red signal, which detects, probe is seen. There was no incorporation of any IdU or CldU labels. (E). pBSpuroG molecules were detected using probe directed against G fragment. Labeling of entire G region with CldU shows that transition site is in G region. Merge panel shows that same molecule was visualized. pBSpuro molecules were detected using probe directed against the puromycin resistance gene. For D-E, rows are (top to bottom): IdU, CldU, probe, and merge.

FIG. 8. A. Graphic representation of the sequence of AT regions of G and control region using Vector NTI software. B. Detection of ORC2 and MCM3 in chromatin immunoprecipitated from 293 cells transfected with pBSpuroG, pBSpuroAT, pBSpuroK5 and pBSpuro. C. Control AT region does not bind to cellular replication machinery. Western blot showing IP of chromatin with anti-ORC2 and anti-MCM3 from pBSpuro cAT- and PBSpuro gAT-transfected 293 cells. Amplification of adjacent puromycin gene target shows absence of amplification with both ORC2 and MCM3 antibodies. By contrast, gAT region containing plasmid was immunoprecipitated with both the antibodies.

### DETAILED DESCRIPTION OF THE INVENTION

This invention provides methods of reducing a replication of a gammaherpesvirus genome, treating a KSHV infection, and treating or reducing an incidence of a KSHV-associated disease, comprising contacting a subject with a composition that inhibits initiation of DNA replication from a region of a genome of a gammaherpesvirus. The invention also provides isolated DNA molecules capable of episomal replication in a eukaryotic cell, and methods of delivering a recombinant protein or therapeutic RNA molecule, comprising same.

In one embodiment, the present invention provides a method of reducing a replication of a genome of a gammaherpesvirus in a subject, comprising contacting the subject with a composition that inhibits initiation of DNA replication from a region of a genome of the gammaherpesvirus, thereby reducing a replication of a genome of a gammaherpesvirus in a subject. In another embodiment, the sequence of the region is set forth in SEQ ID No: 2. In another embodiment, the sequence of the region is a fragment of SEQ ID No: 2. In another embodiment, the region is homologous to SEQ ID No: 2. In another embodiment, the region is homologous to a fragment of SEQ ID No: 2. Each possibility represents a separate embodiment of the present invention.

In another embodiment, the present invention provides a method of reducing a replication of a genome of a gammaherpesvirus in a subject, comprising contacting the subject with a composition that inhibits initiation of DNA replication from a region of a genome of the gammaherpesvirus, wherein the sequence of the region is a fragment of SEQ ID No: 2, and wherein the fragment comprises SEQ ID No: 5, thereby reducing a replication of a genome of a gammaherpesvirus in a subject.

In another embodiment, the present invention provides a method of reducing a replication of a genome of a gammaherpesvirus in a subject, comprising contacting the subject with a composition that inhibits an initiation of DNA replication from a region of a genome of the gammaherpesvirus, wherein the sequence of the region is set forth in SEQ ID No: 5, thereby reducing a replication of a genome of a gammaherpesvirus in a subject.

As provided herein, fragment G of the KSHV genome contains a LANA-independent functional origin of replication in its AT region that mediates long-term maintenance of an episomal KSHV genome in eukaryotic cells (Examples). Thus, inhibiting replication from the region reduces viral replication.

In another embodiment, a G fragment of the present invention has the sequence set forth in SEQ ID No: 1. In another embodiment, the sequence of the G fragment is:

(SEQ ID No: 16)

ggagggggatcccggcgcgccaccctccccggcaacaacctgttgccatg
tatggcgatttgtatcagtcacaagcacacaacccctgctagtattaatg
gtgtttaaaacgttctacacgtacggcggaccgcatccgtcgcaagcacg
cgcatataacccccaaatgcaccatgatgagaagcacagccacgcgtcaa
aaaactttaaaaacatcgttatccaatatcattaaaaaccacaccgaaat
ttacacaggtagcacgtcaccgtgttagtgtcacccactgtacacaaggc
gtgtcgtatatgtagtataggtatttgatgaggcggaagcatatcccgct
tccagcgaacggaaataagaatcatccgttccagcatttattcaaagagg
gcacagaggattcacattgtttagagagagtttttcttagtcaccattcc
atacttgggcagtattggcctacgatttgggcgacgtttcaggctggtct
attctccgtccacttttccccggctattctgtcccagcataggctcttga
aataaacaatgtttaccgagtaaaaggttccactcaccctcatttgtcgt
tgcacccatcccccctttgcttaatcacccgaaaactagaggacacggat
ggaaaacatatcgcacgcgggttgtttgaaagtcaacagctacttgtttt
taatgaggacagatttgggcacaggccagagggtaaagccctacgtgtgc
gcgggggggggggtgtatacgctgcgaaaacctgcacggtgcataacacc
cagggcgtcacgtcacatatctctgtgcacccaagtggttgttcaaccgt
tgttttttggatgatttttccgcaccggcttttttgtgggcgcgcatagg
tcggtacgcgctgtccccctaagtcccgcacggtcgttcgggcccccgtc
cggctcgtctccggatgaaccgtcacgttctttgtctccagaggcgacgt
ctccttcagatgactcgtccgtgggctcctcgtccgtcccgcccgcgggt
ccgacaaggaccgtcaattcgatgttatcttcgttcgcggttggccggcg
cggccgtcggtatggcagtacggtcacccgggtgttatttgccgcgtata
atgccctcacagtgccacttacgcggcatatgccgccaaatgcaaacaca
ataaatatttggtaaaacccaaagaagcagagaaaaccgagcacggcccc
gggggagaatgttcccgcaggagcagttaggatgaccaggagcgtccagg
tgcacaacgccacgccgacaagcccagccaccaccacagacatcagcaga
aacagttcaaaaatttcttggcgctccatctccggccacaggttaaggcg
actacgccactgcgtgcgcgtgcggtatataacgcgacacatttgacagg
ccgtgtttcgagacactgttagccaagtgcttaaacactgcgggtggacg
acatccagctctccggtacaggcgcaggggtgtatgccctcgttccccac
ctcttccctacatatccagcagatgggtccctctacaccctcttctacgt
ccttagacgccatctctgcagctggggtggaagtctgaaaaagggaaagg
ggaggtgagcagagtgcccagttagtctccgacccgccgtccgccctact
gtcgctatcccgccttgacagatgtctaacgtattcacggacgccacatg
tgtgtctattttcctacatccaggctttccctggaaaactgtcacaaccc
accctgctttagctctacatctgtatttttgtttacgcacaggatcaacg
cttcgtgcccgtccacccccgcgctctccgcctgtgtttggaggttttat
gagtggttagttctaggcagctccggacaagttgtccaaaacacggcgcg
ccccgcccttccttccctcc.

In another embodiment, the G fragment is any other KSHV G fragment known in the art. Each possibility represents a separate embodiment of the present invention.

In another embodiment, the gammaherpesvirus is a rhadinovirus. In another embodiment, the gammaherpesvirus is a lymphocryptovirus. In another embodiment, the gammaherpesvirus is any other gammaherpesvirus known in the art. Each possibility represents a separate embodiment of the present invention.

In another embodiment, the rhadinovirus that is targeted by a method of the present invention is a Kaposi's Sarcoma-Associated Herpesvirus (KSHV). In another embodiment, the rhadinovirus is herpesvirus saimiri (HVS) In another embodiment, the gammaherpesvirus is rhesus rhadinovirus (RRV). In another embodiment, the gammaherpesvirus is any other gammaherpesvirus known in the art. Each possibility represents a separate embodiment of the present invention.

In another embodiment, the replication that is reduced or inhibited by a method of the present invention is a LANA-independent replication. In another embodiment, the replication is a latent replication. “Latent replication” refers, in another embodiment, to replication that occurs in a latently infected cell. In another embodiment, a method of the present invention reduces or inhibits total replication of the virus. In another embodiment, the subject has been contacted with, or is receiving, a therapy that targets LANA or LANA-dependent replication. In another embodiment, the therapy is anti-LANA immunotherapy. In another embodiment, the therapy comprises an inhibitor of LANA. In another embodiment, the subject is mounting an immune response against LANA, such that some or all LANA-expressing cells are killed, inactivated, or otherwise selected against. Each possibility represents a separate embodiment of the present invention.

Since LANA is a highly immunogenic protein, the host immune system is likely to recognize infection by detection of LANA, thus inhibiting viral propagation. Once KSHV infects a host B or endothelial cells, however, it can persist indefinitely, reactivating only if immunity is compromised. The autonomous replicating elements of the present invention play a functional role in replication of the KSHV genome, maintaining viral DNA at a desired copy numbers in immune-competent patients. In another embodiment, therapies that target these elements eliminate latent viral replication, allowing clearance of KSHV from the body. In another embodiment, a therapy that targets these elements effectively eliminate KSHV infection in combination with anti-LANA therapy or an anti-LANA immune response. In another embodiment, such a therapy reduces viral load in combination with anti-LANA therapy or an anti-LANA immune response. In another embodiment, the subject of a method of the present invention is a subject wherein LANA-independent replication does not occur. In another embodiment, LANA-independent replication occurs in only a minority of infected cells in the subject. Each possibility represents a separate embodiment of the present invention.

Describing initiation of DNA replication as “from a [specified] region” of a viral genome refers, in another embodiment, to replication that utilizes the specified region to bind or interact with a component of the cellular DNA replication machinery. In another embodiment, the terms refers to replication that is initiated in the specified region. In another embodiment, the replication is initiated in the vicinity of the specified region (e.g. in various embodiments, within 10 base pairs (bp), 15 bp, 20 bp, 25 bp, 30 bp, 40 bp, 60 bp, 80 bp, 100 bp, 120 bp, 140 bp, 160 bp, 200 bp, 250 bp, 300 bp, 350 bp, 400 bp, 500 bp, 600 bp, 800 bp, 1 kilobase pairs [kbp], 1.5 kbp, 2 kbp, 2.5 kbp, 3 kbp, 4 kbp, 6 kbp, 8 kbp, or 10 kbp). Each possibility represents a separate embodiment of the present invention.

The viral genome that is targeted by a method of the present invention is, in another embodiment, episomal. In another embodiment, the viral genome is a circular DNA molecule. In another embodiment, the viral genome is extra-chromosomal. In another embodiment, the viral genome is a gammaherpesvirus genome. In another embodiment, the viral genome is any other type of viral genome known in the art. Each possibility represents a separate embodiment of the present invention.

The composition utilized in methods of the present invention comprises, in another embodiment, a small molecule inhibitor of replication from a genomic region of the present invention. In another embodiment, the composition comprises a peptide nucleic acid (PNA). In another embodiment, the composition comprises a nucleotide or deoxynucleotide. In another embodiment, the composition comprises any other type of substance known in the art that can interact with a DNA molecule or cellular DNA replication machinery. In another embodiment, the composition comprises one or more of the inactive ingredients, excipients, etc, that are enumerated below. Each possibility represents a separate embodiment of the present invention.

In another embodiment, the composition utilized in a method of the present invention or a component thereof interacts with the region of the genome that is targeted by a method of the present invention. In another embodiment, the composition binds the region. In another embodiment, the composition interacts with the cellular DNA replication machinery. In another embodiment, the composition binds the cellular DNA replication machinery. In another embodiment, the composition inhibits binding of a DNA replication protein to the region. Each possibility represents a separate embodiment of the present invention.

In another embodiment, the present invention provides a method of treating a KSHV infection in a subject, comprising contacting the subject with a composition of the present invention, thereby treating a KSHV infection in a subject.

In another embodiment, the present invention provides a method of treating a KSHV-associated Kaposi's sarcoma (KS) in a subject, comprising contacting the subject with a composition of the present invention, thereby treating a KSHV-associated KS in a subject.

In another embodiment, the present invention provides a method of reducing an incidence of a KSHV-associated KS in a subject, comprising contacting the subject with a composition of the present invention, thereby reducing an incidence of a KSHV-associated KS in a subject.

In another embodiment, the present invention provides a method of treating a KSHV-associated primary effusion lymphoma (PEL) in a subject, comprising contacting the subject with a composition of the present invention, thereby treating a KSHV-associated PEL in a subject.

In another embodiment, the present invention provides a method of reducing an incidence of a KSHV-associated PEL in a subject, comprising contacting the subject with a composition of the present invention, thereby reducing an incidence of a KSHV-associated PEL in a subject.

In another embodiment, the present invention provides a method of treating a KSHV-associated multicentric Castleman's disease in a subject, comprising contacting the subject with a composition of the present invention, thereby treating a KSHV-associated multicentric Castleman's disease in a subject.

In another embodiment, the present invention provides a method of reducing an incidence of a KSHV-associated multicentric Castleman's disease in a subject, comprising contacting the subject with a composition of the present invention, thereby reducing an incidence of a KSHV-associated multicentric Castleman's disease in a subject.

In another embodiment, the present invention provides an isolated DNA molecule, comprising (a) a non-Kaposi's Sarcoma-Associated Herpesvirus (KSHV) portion; and (b) a region of a KSHV genome.

In another embodiment, the sequence of the region is set forth in SEQ ID No: 2. In another embodiment, the sequence of the region is a fragment of SEQ ID No: 2. In another embodiment, the region is homologous to SEQ ID No: 2. In another embodiment, the region is homologous to a fragment of SEQ ID No: 2. In another embodiment, the sequence of the region consists of SEQ ID No: 2. In another embodiment, the sequence of the region comprises SEQ ID No: 5. In another embodiment, the sequence of the region consists of SEQ ID No: 5. In another embodiment, the isolated DNA molecule is capable of episomal replication in a eukaryotic cell. Each possibility represents a separate embodiment of the present invention.

In another embodiment, the present invention provides an isolated DNA molecule, comprising a region of a KSHV genome, wherein the region is a KSHV fragment of the present invention. In another embodiment, the sequence of the region is set forth in SEQ ID No: 2. In another embodiment, the sequence of the region is a fragment of SEQ ID No: 2. In another embodiment, the region is homologous to SEQ ID No: 2. In another embodiment, the region is homologous to a fragment of SEQ ID No: 2. In another embodiment, the sequence of the region consists of SEQ ID No: 2. In another embodiment, the sequence of the region comprises SEQ ID No: 5. In another embodiment, the region has a sequence consisting of SEQ ID No: 5. In another embodiment, the isolated DNA molecule is capable of episomal replication in a eukaryotic cell. Each possibility represents a separate embodiment of the present invention.

In another embodiment, the present invention provides a gene therapy vector, comprising (a) a gene encoding a protein of interest; and (b) a region of a KSHV genome. In another embodiment, the sequence of the region is set forth in SEQ ID No: 2. In another embodiment, the sequence of the region is a fragment of SEQ ID No: 2. In another embodiment, the region is homologous to SEQ ID No: 2. In another embodiment, the region is homologous to a fragment of SEQ ID No: 2. In another embodiment, the sequence of the region consists of SEQ ID No: 2. In another embodiment, the sequence of the region comprises SEQ ID No: 5. In another embodiment, the sequence of the region consists of SEQ ID No: 5. In another embodiment, the gene therapy vector is capable of episomal replication in a eukaryotic cell. In another embodiment, the gene therapy vector is a non-integrating vector. In another embodiment, the protein of interest is a therapeutic protein. Each possibility represents a separate embodiment of the present invention.

In another embodiment, the present invention provides a gene therapy vector, comprising (a) a gene encoding a therapeutic RNA molecule; and (b) a region of a KSHV genome. In another embodiment, the sequence of the region is set forth in SEQ ID No: 2. In another embodiment, the sequence of the region is a fragment of SEQ ID No: 2. In another embodiment, the region is homologous to SEQ ID No: 2. In another embodiment, the region is homologous to a fragment of SEQ ID No: 2. In another embodiment, the sequence of the region consists of SEQ ID No: 2. In another embodiment, the sequence of the region comprises SEQ ID No: 5. In another embodiment, the sequence of the region consists of SEQ ID No: 5. In another embodiment, the gene therapy vector is capable of episomal replication in a eukaryotic cell. Each possibility represents a separate embodiment of the present invention.

In another embodiment, an isolated DNA molecule or gene therapy vector of the present invention further comprises a recombinant gene. In another embodiment, the recombinant gene encodes a therapeutic protein. In another embodiment, the recombinant gene encodes a therapeutic RNA molecule. Each possibility represents a separate embodiment of the present invention.

In another embodiment, an isolated DNA molecule or gene therapy vector of methods and compositions of the present invention is capable of generating self copies. In another embodiment, the self copies exhibit long-term persistence in the eukaryotic cell and descendents therefrom. Each possibility represents a separate embodiment of the present invention.

“Self copies” refers, in another embodiment to copies of the isolated DNA molecule. In another embodiment, the term refers to copies of a larger DNA molecule that contains the isolated DNA molecule. In another embodiment, the term refers to copies of a fragment of the isolated DNA molecule. In another embodiment, the term refers to copies of a fragment that comprises the sequence set forth in SEQ ID No: 2. Each possibility represents a separate embodiment of the present invention.

“Capable of generating” self copies refers, in another embodiment, to a DNA molecule that is capable of self-replication in the target cell. In another embodiment, the DNA molecule is capable of self-replication in a eukaryotic cell. In another embodiment, the DNA molecule is capable of self-replication in a particular subset of eukaryotic cells. In another embodiment, the DNA molecule is capable of generation of self copies (e.g. as defined above). Each possibility represents a separate embodiment of the present invention.

In other embodiments, the isolated DNA molecules of any of the methods of the present invention can have any of the characteristics of an isolated DNA molecule of the present invention. Each characteristic represents a separate embodiment of the present invention.

In another embodiment, the present invention provides a method of delivering a recombinant protein to a subject, comprising administering to the subject a DNA molecule or gene therapy vector of the present invention, thereby delivering a recombinant protein to a subject. In another embodiment, the DNA molecule further comprises a recombinant gene. The recombinant gene, in another embodiment, encodes the recombinant protein to be delivered. In another embodiment, the recombinant protein is a therapeutic protein. Each possibility represents a separate embodiment of the present invention.

In another embodiment, a method of the present invention of delivering a recombinant protein or therapeutic RNA molecule is performed in vivo. In another embodiment, the method is performed ex vivo (e.g. isolated cells are contacted with the DNA molecule of the present invention, then administered to the subject). In another embodiment, the isolated cells are from the subject. In another embodiment, the cells are from a donor. In another embodiment, the cells are amplified ex vivo. Each possibility represents a separate embodiment of the present invention.

In another embodiment, the present invention provides a method of delivering a therapeutic RNA molecule to a subject, comprising administering to the subject a DNA molecule or gene therapy vector of the present invention, wherein the DNA molecule further comprises a recombinant gene, the recombinant gene encoding the therapeutic RNA molecule, thereby delivering a therapeutic RNA molecule to a subject. In another embodiment, a KSHV replication element of the present invention is used as a replicon origin in the DNA molecule. In another embodiment, the replication element can support replication in human cells. In another embodiment, the replication element enables long-term persistence of the DNA molecule. Each possibility represents a separate embodiment of the present invention.

In another embodiment, a therapeutic RNA molecule further comprises an open reading frame that encodes a functional protein. In another embodiment, the therapeutic RNA molecule functions without encoding a functional protein. In another embodiment, the therapeutic RNA molecule functions by transcriptional silencing. In another embodiment, the therapeutic RNA molecule functions as an RNzyme. In another embodiment, the therapeutic RNA molecule is an antisense molecule. In another embodiment, the therapeutic RNA molecule is an siRNA molecule. In another embodiment, the therapeutic RNA molecule is a micro RNA molecule. In another embodiment, the therapeutic RNA molecule is any other type of inhibitory RNA molecule known in the art. Each possibility represents a separate embodiment of the present invention

As provided herein, a LANA-independent functional origin of replication in the KSHV genome has been identified (Examples). This origin of replication can be used in various types of gene therapy vectors to confer persistence in target cells and the descendants, using techniques well known in the art. In addition, since the origin mediates episomal replication, it can be used in a non-integrating gene therapy vector. In another embodiment, a non-integrating gene therapy vector of the present invention exhibits enhanced safety over an integrating vector. Each possibility represents a separate embodiment of the present invention.

In other embodiments, the DNA molecule contains a gene that encodes one of the following proteins: ABCA4; ABCD3; ACADM; AGL; AGT; ALDH4A1; ALPL; AMPD1; APOA2; AVSD1; BRCD2; C1QA; C1QB; C1QG; C8A; C8B; CACNA1S; CCV; CD3Z; CDC2L1; CHML; CHS1; CIAS1; CLCNKB; CMD1A; CMH2; CMM; COL11A1; COL8A2; COL9A2; CPT2; CRB1; CSE; CSF3R; CTPA; CTSK; DBT; DIO1; DISC1; DPYD; EKV; ENO1; ENO1P; EPB41; EPHX1; F13B; F5; FCGR2A; FCGR2B; FCGR3A; FCHL; FH; FMO3; FMO4; FUCA1; FY; GALE; GBA; GFND; GJA8; GJB3; GLC3B; HF1, HMGCL; HPC1; HRD; HRPT2; HSD3B2; HSPG2; KCNQ4; KCS; KIF1B; LAMB3; LAMC2; LGMD1B; LMNA; LOR; MCKD1; MCL1; MPZ; MTHFR; MTR; MUTYH; MYOC; NB; NCF2; NEM1; NPHS2; NPPA; NRAS; NTRK1; OPTA2; PBX1; PCHC; PGD; PHA2A; PHGDH; PKLR; PKP1; PLA2G2A; PLOD; PPOX; PPT1; PRCC; PRG4; PSEN2; PTOS1; REN; RFX5; RHD; RMD1; RPE65; SCCD; SERPINC1; SJS1; SLC19A2; SLC2A1; SPG23; SPTA1; TAL1; TNFSF6; TNNT2; TPM3; TSHB; UMPK; UOX; UROD; USH2A; VMGLOM; VWS; WS2B; ABCB11; ABCG5; ABCG8; ACADL; ACP1; AGXT; AHHR; ALMS1; ALPP; ALS2; APOB; BDE; BDMR; BJS; BMPR2; CHRNA1; CMCWTD; CNGA3; COL3A1; COL4A3; COL4A4; COL6A3; CPS1; CRYGA; CRYGEP1; CYPiB1; CYP27A1; DBI; DES; DYSF; EDAR; EFEMP1; EIF2AK3; ERCC3; FSHR; GINGF; GLC1B; GPD2; GYPC; HADHA; HADHB; HOXD13; HPE2; IGKC; IHH; IRS1; ITGA6; KHK; KYNU; LCT; LHCGR; LSFC; MSH2; MSH6; NEB; NMTC; NPHP1; PAFAH1P1; PAX3; PAX8; PMS1; PNKD; PPH1; PROC; REG1A; SAG; SFTPB; SLC11A1; SLC3A1; SOS1; SPG4; SRD5A2; TCL4; TGFA; TMD; TPO; UGT1A@; UV24; WSS; XDH; ZAP70; ZFHX1B; ACAA1; AGS1; AGTR1; AHSG; AMT; ARMET; BBS3; BCHE; BCPM; BTD; CASR; CCR2; CCR5; CDL1; CMT2B; COL7A1; CP; CPO; CRV; CTNNBl; DEM; ETM1; FANCD2; FIH; FOXL2; GBE1; GLB1; GLC1C; GNAI2; GNAT1; GP9; GPX1; HGD; HRG; ITIH1; KNG; LPP; LRS1; MCCC1; MDS1; MHS4; MITF; MLH1; MYL3; MYMY; OPA1; P2RY12; PBXP1; PCCB; POU1F1; PPARG; PROS1; PTHR1; RCA1; RHO; SCA7; SCLC1; SCN5A; SI; SLC25A20; SLC2A2; TF; TGFBR2; THPO; THRB; TKT; TM4SF1; TRH; UMPS; UQCRC1; USH3A; VHL; WS2A; XPC; ZNF35; ADH1B; ADH1C; AFP; AGA; AIH2; ALB; ASMD; BFHD; CNGA1; CRBM; DCK; DSPP; DTDP2; ELONG; ENAM; ETFDH; EVC; F11; FABP2; FGA; FGB; FGFR3; FGG; FSHMD1A; GC; GNPTA; GNRHR; GYPA; HCA; HCL2; HD; HTN3; HVBS6; IDUA; IF; JPD; KIT; KLKB1; LQT4; MANBA; MLLT2; MSX1; MTP; NR3C2; PBT; PDE6B; PEE1; PITX2; PKD2; QDPR; SGCB; SLC25A4; SNCA; SOD3; STATH; TAPVR1; TYS; WBS2; WFS1; WHCR; ADAMTS2; ADRB2; AMCN; AP3B1; APC; ARSB; B4GALT7; BHR1; C6; C7; CCAL2; CKN1; CMDJ; CRHBP; CSF1R; DHFR; DIAPH1; DTR; EOS; EPD; ERVR; F12; FBN2; GDNF; GHR; GLRA1; GM2A; HEXB; HSD17B4; ITGA2; KFS; LGMD1A; LOX; LTC4S; MAN2A1; MCC; MCCC2; MSH3; MSX2; NR3C1; PCSK1; PDE6A; PFBI; RASA1; SCZD1; SDHA; SGCD; SLC22A5; SLC26A2; SLC6A3; SM1; SMA@; SMN1; SMN2; SPINK5; TCOF1; TELAB1; TGFBI; ALDH5A1; ARG1; AS; ASSP2; BCKDHB; BF; C2; C4A; CDKN1A; COL10A1; COL11A2; CYP21A2; DYX2; EJM1; ELOVL4; EPM2A; ESR1; EYA4; F13A1; FANCE; GCLC; GJA1; GLYS1; GMPR; GSE; HCR; HFE; HLA-A; HLA-DPB1; HLA-DRA; HPFH; ICS1; IDDM1; IFNGR1; IGAD1; IGF2R; ISCW; LAMA2; LAP; LCA5; LPA; MCDR1; MOCS1; MUT; MYB; NEU1; NKS1; NYS2; OA3; ODDD; OFC1; PARK2; PBCA; PBCRA1; PDB1; PEX3; PEX6; PEX7; PKHD1; PLA2G7; PLG; POLH; PPAC; PSORS1; PUJO; RCD1; RDS; RHAG; RP14; RUNX2; RWS; SCA1; SCZD3; SIASD; SOD2; ST8; TAP1; TAP2; TFAP2B; TNDM; TNF; TPBG; TPMT; TULP1; WISP3; AASS; ABCB1; ABCB4; ACHE; AQP1; ASL; ASNS; AUTS1; BPGM; BRAF; C7orf2; CACNA2D1; CCM1; CD36; CFTR; CHORDOMA; CLCN1; CMH6; CMT2D; COL1A2; CRS; CYMD; DFNA5; DLD; DYT11; EEC1; ELN; ETV1; FKBP6; GCK; GHRHR; GHS; GLI3; GPDS1; GUSB; HLXB9; HOXA13; HPFH2; HRX; IAB; IMMP2L; KCNH2; LAMB1; LEP; MET; NCF1; NM; OGDH; OPN1SW; PEX1; PGAM2; PMS2; PON1; PPP1R3A; PRSS1; PTC; PTPN12; RP10; RP9; SERPINE1; SGCE; SHFM1; SHH; SLC26A3; SLC26A4; SLOS; SMAD1; TBXAS1; TWIST; ZWS1; ACHM3; ADRB3; ANK1; CA1; CA2; CCAL1; CLN8; CMT4A; CNGB3; COH1; CPP; CRH; CYP11B1; CYP11B2; DECR1; DPYS; DURS1; EBS1; ECA1; EGI; EXT1; EYA1; FGFR1; GNRH1; GSR; GULOP; HR; KCNQ3; KFM; KWE; LGCR; LPL; MCPH1; MOS; MYC; NAT1; NAT2; NBS1; PLAT; PLEC1; PRKDC; PXMP3; RP1; SCZD6; SFTPC; SGM1; SPG5A; STAR; TG; TRPS1; TTPA; VMD1; WRN; ABCA1; ABL1; ABO; ADAMTS13; AK1; ALAD; ALDH1A1; ALDOB; AMBP; AMCD1; ASS; BDMF; BSCL; C5; CDKN2A; CHAC; CLA1; CMD1B; COL5A1; CRAT; DBH; DNAI1; DYS; DYT1; ENG; FANCC; FBP1; FCMD; FRDA; GALT; GLDC; GNE; GSM1; GSN; HSD17B3; HSN1; IBM2; INVS; JBTS1; LALL; LCCS1; LCCS; LGMD2H; LMX1B; MLLT3; MROS; MSSE; NOTCH1; ORM1; PAPPA; PIP5K1B; PTCH; PTGS1; RLN1; RLN2; RMRP; ROR2; RPD1; SARDH; SPTLC1; STOM; TDFA; TEK; TMC1; TRIM32; TSC1; TYRP1; XPA; CACNB2; COL17A1; CUBN; CXCL12; CYP17; CYP2C19; CYP2C9; EGR2; EMX2; ERCC6; FGFR2; HK1; HPS1; IL2RA; LGI1; L1PA; MAT1A; MBL2; MKI67; MXI1; NODAL; OAT; OATL3; PAX2; PCBD; PEO1; PHYH; PNLIP; PSAP; PTEN; RBP4; RDPA; RET; SFTPA1; SFTPD; SHFM3; SIAL; THC2; TLX1; TNFRSF6; UFS; UROS; AA; ABCC8; ACAT1; ALX4; AMPD3; ANC; APOA1; APOA4; APOC3; ATM; BSCL2; BWS; CALCA; CAT; CCND1; CD3E; CD3G; CD59; CDKN1C; CLN2; CNTF; CPT1A; CTSC; DDB1; DDB2; DHCR7; DLAT; DRD4; ECB2; ED4; EVR1; EXT2; F2; FSHB; FTH1; G6PT1; G6PT2; GIF; HBB; HBBP1; HBD; HBE1; HBG1; HBG2; HMBS; HND; HOMG2; HRAS; HVBS1; IDDM2; IGER; INS; JBS; KCNJ11; KCNJ1; KCNQ1; LDHA; LRP5; MEN1; MLL; MYBPC3; MYO7A; NNO1; OPPG; OPTB1; PAX6; PC; PDX1; PGL2; PGR; PORC; PTH; PTS; PVRL1; PYGM; RAG1; RAG2; ROM1; RRAS2; SAA1; SCA5; SCZD2; SDHD; SERPING1; SMPD1; TCIRG1; TCL2; TECTA; TH; TREH; TSG101; TYR; USH1C; VMD2; VRNI; WT1; WT2; ZNF145; A2M; AAAS; ACADS; ACLS; ACVRL1; ALDH2; AMHR2; AOM; AQP2; ATD; ATP2A2; BDC; C1R; CD4; CDK4; CNA1; COL2A1; CYP27B1; DRPLA; ENUR2; FEOM1; FGF23; FPF; GNB3; GNS; HAL; HBP1; HMGA2; HMN2; HPD; IGF1; KCNA1; KERA; KRAS2; KRT1; KRT2A; KRT3; KRT4; KRT5; KRT6A; KRT6B; KRTHB6; LDHB; LYZ; MGCT; MPE; MVK; MYL2; OAP; PAH; PPKB; PRB3; PTPNl1; PXR1; RLS; RSN; SAS; SAX1; SCA2; SCNN1A; SMAL; SPPM; SPSMA; TBX3; TBX5; TCF1; TP11; TSC3; ULR; VDR; VWF; ATP7B; BRCA2; BRCD1; CLN5; CPB2; ED2; EDNRB; ENUR1; ERCC5; F10; F7; GJB2, GJB6; IPF1; MBS1; MCOR; NYS4; PCCA; RB1; RHOK; SCZD7; SGCG; SLC10A2; SLC25A15; STARP1; ZNF198; ACHM1; ARVD1; BCH; CTAA1; DAD1; DFNB5; EML1; GALC; GCH1; IBGC1; IGH@; IGHC group; IGHG1; IGHM; IGHR; IV; LTBP2; MCOP; MJD; MNG1; MPD1; MPS3C; MYH6; MYH7; NP; NPC2; PABPN1; PSEN1; PYGL; RPGRIP1; SERPINA1; SERPINA3; SERPINA6; SLC7A7; SPG3A; SPTB; TCL1A; TGM1; TITF1; TMIP; TRA@; TSHR; USH1A; VP; ACCPN; AHO2; ANCR; B2M; BBS4; BLM; CAPN3; CDAN1; CDAN3; CLN6; CMH3; CYP19; CYP1A1; CYP1A2; DYX1; EPB42; ETFA; EYCL3; FAH; FBN1; FES; HCVS; HEXA; IVD; LCS1; LIPC; MYO5A; OCA2; OTSC1; PWCR; RLBP1; SLC12A1; SPG6; TPM1; UBE3A; WMS; ABCC6; ALDOA; APRT; ATP2A1; BBS2; CARD15; CATM; CDH1; CETP; CHST6; CLN3; CREBBP; CTH; CTM; CYBA; CYLD; DHS; DNASE1; DPEP1; ERCC4; FANCA; GALNS; GAN; HAGH; HBA1; HBA2; HBHR; HBQ1; HBZ; HBZP; HP; HSD11B2; IL4R; LIPB; MC1R; MEFV; MHC2TA; MLYCD; MMVP1; PHKB; PHKG2; PKD1; PKDTS; PMM2; PXE; SALL1; SCA4; SCNN1B; SCNN1G; SLC12A3; TAT; TSC2; VDI; WT3; ABR; ACACA; ACADVL; ACE; ALDH3A2; APOH; ASPA; AXIN2; BCL5; BHD; BLMH; BRCA1; CACD; CCA1; CCZS; CHRNB1; CHRNE; CMT1A; COL1A1; CORD5; CTNS; EPX; ERBB2; G6PC; GAA; GALK1; GCGR; GFAP; GH1; GH2; GP1BA; GPSC; GUCY2D; ITGA2B; ITGB3; ITGB4; KRT10; KRT12; KRT13; KRT14; KRT14L1; KRT14L2; KRT14L3; KRT16; KRT16L1; KRT16L2; KRT17; KRT9; MAPT; MDB; MDCR; MGI; MHS2; MKS1; MPO; MYO15A; NAGLU; NAPB; NF1; NME1; P4HB; PAFAH1B1; PECAM1; PEX12; PHB; PMP22; PRKAR1A; PRKCA; PRKWNK4; PRP8; PRPF8; PTLAH; RARA; RCV1; RMSA1; RP17; RSS; SCN4A; SERPINF2; SGCA; SGSH; SHBG; SLC2A4; SLC4A1; SLC6A4; SMCR; SOST; SOX9; SSTR2; SYM1; SYNS1; TCF2; THRA; TIMP2; TOC; TOP2A; TP53; TRIM37; VBCH; ATP8B1; BCL2; CNSN; CORD1; CYB5; DCC; F5F8D; FECH; FEO; LAMA3; LCFS2; MADH4; MAFD1; MC2R; MCL; MYP2; NPC1; SPPK; TGFBRE; TGIF; TTR; AD2; AMH; APOC2; APOE; ATHS; BAX; BCKDHA; BCL3; BFIC; C3; CACNA1A; CCO; CEACAM5; COMP; CRX; DBA; DDU; DFNA4; DLL3; DM1; DMWD; E11S; ELA2; EPOR; ERCC2; ETFB; EXT3; EYCL1; FTL; FUT1; FUT2; FUT6; GAMT; GCDH; GPI; GUSM; HB1; HCL1; HHC2; HHC3; ICAM3; INSR; JAK3; KLK3; LDLR; LHB; LIG1; LOH19CR1; LYL1; MAN2B1; MCOLN1; MDRV; MLLT1; NOTCH3; NPHS1; OFC3; OPA3; PEPD; PRPF31; PRTN3; PRX; PSG1; PVR; RYR1; SLC5A5; SLC7A9; STK11; TBXA2R; TGFB1; TNNI3; TYROBP; ADA; AHCY; AVP; CDAN2; CDPD1; CHED1; CHED2; CHRNA4; CST3; EDN3; EEGV1; FTLL1; GDF5; GNAS; GSS; HNF4A; JAG1; KCNQ2; MKKS; NBIA1; PCK1; PI3; PPCD; PPGB; PRNP; THBD; TOP1; AIRE; APP; CBS; COL6A1; COL6A2; CSTB; DCR; DSCR1; FPDMM; HLCS; HPE1; ITGB2; KCNE1; KNO; PRSS7; RUNX1; SOD1; TAM; ADSL; ARSA; BCR; CECR; CHEK2; COMT; CRYBB2; CSF2RB; CTHM; CYP2D6; CYP2D7P1; DGCR; DIA1; EWSR1; GGT1; MGCR; MN1; NAGA; NF2; OGS2; PDGFB; PPARA; PRODH; SCO2; SCZD4; SERPIND1; SLC5A1; SOX10; TCN2; TIMP3; TST; VCF; ABCD1; ACTL1; ADFN; AGMX2; AHDS; AIC; AIED; AIH3; ALAS2; AMCD; AMELX; ANOP1; AR; ARAF1; ARSC2; ARSE; ARTS; ARX; ASAT; ASSP5; ATP7A; ATRX; AVPR2; BFLS; BGN; BTK; BZX; C1HR; CACNALF; CALB3; CBBM; CCT; CDR1; CFNS; CGF1; CHM; CHR39c; CIDX; CLA2; CLCN5; CLS; CMTX2; CMTX3; CND; COD1; COD2; COL4A5; COL4A6; CPX; CVD1; CYBB; DCX; DFN2; DFN4; DFN6; DHOF; DIAPH2; DKC1; DMD; DSS; DYT3; EBM; EBP; ED1; ELK1; EMD; EVR2; F8; F9; FCP1; FDPSL5; FGD1; FGS1; FMR1; FMR2; G6PD; GABRA3; GATA1; GDI1; GDXY; GJB1; GK; GLA; GPC3; GRPR; GTD; GUST; HMS1; HPRT1; HPT; HTC2; HTR2C; HYR; IDS; IHG1; IL2RG; INDX; IP1; IP2; JMS; KAL1; KFSD; L1CAM; LAMP2; MAA; MAFD2; MAOA; MAOB; MCF2; MCS; MEAX; MECP2; MF4; MGC1; MIC5; MID1; MLLT7; MLS; MRSD; MRX14; MRX1; MRX20; MRX2; MRX3; MRX40; MRXA; MSD; MTM1; MYCL2; MYP1; NDP; NHS; NPHL1; NROB1; NSX; NYS1; NYX; OA1; OASD; OCRL; ODT1; OFD1; OPA2; OPD1; OPEM; OPN1LW; OPN1MW; OTC; P3; PDHA1; PDR; PFC; PFKFB1; PGK1; PGK1P1; PGS; PHEX; PHKA1; PHKA2; PHP; PIGA; PLP1; POF1; POLA; POU3F4; PPMX; PRD; PRPS1; PRPS2; PRS; RCCP2; RENBP; RENS1; RP2; RP6; RPGR; RPS4X; RPS6KA3; RS1; S11; SDYS; SEDL; SERPINA7; SH2D1A; SHFM2; SLC25A5; SMAX2; SRPX; SRS; STS; SYN1; SYP; TAF1; TAZ; TBX22; TDD; TFE3; THAS; THC; TIMM8A; TIMP1; TKCR; TNFSF5; UBE1; UBE2A; WAS; WSN; WTS; WWS; XIC; XIST; XK; XM; XS; ZFX; ZIC3; ZNF261; ZNF41; ZNF6; AMELY; ASSP6; AZF1; AZF2; DAZ; GCY; RPS4Y; SMCY; SRY; ZFY; ABAT; AEZ; AFA; AFD1; ASAH1; ASD1; ASMT; CCAT; CECR9; CEPA; CLA3; CLN4; CSF2RA; CTS1; DF; DIH1; DWS; DYT2; DYT4; EBR3; ECT; EEF1A1L14; EYCL2; FANCB; GCSH; GCSL; GIP; GTS; HHG; HMI; HOAC; HOKPP2; HRPT1; HSD3B3; HTC1; HVLS; ICHQ; ICR1; ICR5; IL3RA; KAL2; KMS; KRT18; KSS; LCAT; LHON; LIMM; MANBB; MCPH2; MEB; MELAS; MIC2; MPFD; MS; MSS; MTATP6; MTCO1; MTCO3; MTCYB; MTND1; MTND2; MTND4; MTND5; MTND6; MTRNR1; MTRNR2; MTTE; MTTG; MTTI; MTTK; MTTL1; MTTL2; MTTN; MTTP; MTTS1; NAMSD; OCD1; OPD2; PCK2; PCLD; PCOS1; PFKM; PKD3; PRCA1; PRO1; PROP1; RBS; RFXAP; RP; SHOX; SLC25A6; SPG5B; STO; SUOX; THM; or TTD. Each recombinant protein represents a separate embodiment of the present invention.

In another embodiment, an isolated DNA molecule of methods and compositions of the present invention is capable of generating copies of self that exhibit long-term persistence in the subject. In another embodiment, this property of the isolated DNA molecule enables delivery of the recombinant protein to continue for several weeks following the step of administering. Each possibility represents a separate embodiment of the present invention.

In another embodiment, the delivery of the recombinant protein continues for several months following administration. In another embodiment, the delivery continues for a time period of several years. In another embodiment, time period is at least 3 months. In another embodiment, the time period is at least 2 weeks. In another embodiment, the time period is at least 3 weeks. In another embodiment, the time period is at least 1 month. In another embodiment, the time period is at least 2 months. In another embodiment, the time period is at least 3 months. In another embodiment, the time period is at least 4 months. In another embodiment, the time period is at least 5 months. In another embodiment, the time period is at least 6 months. In another embodiment, the time period is at least 8 months. In another embodiment, the time period is at least 10 months. In another embodiment, the time period is at least 1 year. In another embodiment, the time period is at least 15 months. In another embodiment, the time period is at least 1½ year. In another embodiment, the time period is at least 2 years. In another embodiment, the time period is at least 3 years. In another embodiment, the time period is longer than 3 years. Each possibility represents a separate embodiment of the present invention.

In another embodiment, a gene therapy vector of the present invention persists for several months following administration. In another embodiment, the vector persists for a time period of several years. In another embodiment, time period is at least 3 months. In another embodiment, the time period is at least 2 weeks. In another embodiment, the time period is at least 3 weeks. In another embodiment, the time period is at least 1 month. In another embodiment, the time period is at least 2 months. In another embodiment, the time period is at least 3 months. In another embodiment, the time period is at least 4 months. In another embodiment, the time period is at least 5 months. In another embodiment, the time period is at least 6 months. In another embodiment, the time period is at least 8 months. In another embodiment, the time period is at least 10 months. In another embodiment, the time period is at least 1 year. In another embodiment, the time period is at least 15 months. In another embodiment, the time period is at least 1½ year. In another embodiment, the time period is at least 2 years. In another embodiment, the time period is at least 3 years. In another embodiment, the time period is longer than 3 years. Each possibility represents a separate embodiment of the present invention.

In another embodiment, the present invention provides a composition for reducing a replication of a genome of a gammaherpesvirus in a subject, wherein the composition inhibits an initiation of DNA replication from a region of a genome of the gammaherpesvirus. In another embodiment, the sequence of the region is set forth in SEQ ID No: 2 or a fragment thereof. In another embodiment, the sequence is homologous to SEQ ID No: 2 or a fragment thereof. In another embodiment, the sequence of the fragment comprises SEQ ID No: 5. Each possibility represents a separate embodiment of the present invention.

In another embodiment, the present invention provides a composition for treating a KSHV infection in a subject, wherein the composition inhibits an initiation of DNA replication from a region of a genome of the gammaherpesvirus. In another embodiment, the sequence of the region is set forth in SEQ ID No: 2 or a fragment thereof. In another embodiment, the sequence of the region is homologous to SEQ ID No: 2 or a fragment thereof. In another embodiment, the sequence of the fragment of SEQ ID No 2 comprises SEQ ID No: 5. Each possibility represents a separate embodiment of the present invention.

In another embodiment, the present invention provides a composition for reducing an incidence of a KSHV-associated KS, PEL, or multicentric Castleman's disease in a subject, wherein the composition inhibits an initiation of DNA replication from a region of a genome of the gammaherpesvirus. In another embodiment, the sequence of the region is set forth in SEQ ID No: 2 or a fragment thereof. In another embodiment, the region is homologous to SEQ ID No: 2 or a fragment thereof. In another embodiment, the sequence of the fragment comprises SEQ ID No: 5. Each possibility represents a separate embodiment of the present invention.

In another embodiment, the present invention provides a composition for delivering a recombinant protein to a subject, a composition comprising a DNA molecule, the DNA molecule comprising a region having a sequence set forth in SEQ ID No: 2 or a fragment thereof, and further comprising a recombinant gene encoding the recombinant protein. In another embodiment, the region is homologous to SEQ ID No: 2 or a fragment thereof. In another embodiment, the sequence of the fragment comprises SEQ ID No: 5. In another embodiment, the sequence of the region consists of SEQ ID No: 5. Each possibility represents a separate embodiment of the present invention.

In another embodiment, the present invention provides a composition for delivering a therapeutic RNA molecule to a subject, a composition comprising a DNA molecule, the DNA molecule comprising a region having a sequence set forth in SEQ ID No: 2 or a fragment thereof, and further comprising a recombinant gene encoding the therapeutic RNA molecule. In another embodiment, the region is homologous to SEQ ID No: 2 or a fragment thereof. In another embodiment, the sequence of the fragment comprises SEQ ID No: 5. In another embodiment, the sequence of the region consists of SEQ ID No: 5. Each possibility represents a separate embodiment of the present invention.

In another embodiment, the present invention provides a use of a composition that inhibits initiation of DNA replication from a KSHV genomic fragment of the present invention, in the manufacture of a medicament for reducing a replication of a genome of a gammaherpesvirus in a subject. In another embodiment, the sequence of the region is set forth in SEQ ID No: 2 or a fragment thereof. In another embodiment, the region is homologous to SEQ ID No: 2 or a fragment thereof. In another embodiment, the sequence of the fragment comprises SEQ ID No: 5. Each possibility represents a separate embodiment of the present invention.

In another embodiment, the present invention provides a use of a composition that reduces an intracellular amount of a KSHV genomic fragment of the present invention, in the manufacture of a medicament for reducing a replication of a genome of a gammaherpesvirus in a subject. In another embodiment, the sequence of the region is set forth in SEQ ID No: 2 or a fragment thereof. In another embodiment, the region is homologous to SEQ ID No: 2 or a fragment thereof. In another embodiment, the sequence of the fragment comprises SEQ ID No: 5. Each possibility represents a separate embodiment of the present invention.

In another embodiment, the present invention provides a use of a composition that inhibits initiation of DNA replication from a KSHV genomic fragment of the present invention, in the manufacture of a medicament for treating a KSHV infection in a subject. In another embodiment, the sequence of the region is set forth in SEQ ID No: 2 or a fragment thereof. In another embodiment, the region is homologous to SEQ ID No: 2 or a fragment thereof. In another embodiment, the sequence of the fragment comprises SEQ ID No: 5. Each possibility represents a separate embodiment of the present invention.

In another embodiment, the present invention provides a use of a composition that reduces an intracellular amount of a KSHV genomic fragment of the present invention, in the manufacture of a medicament for treating a KSHV infection in a subject. In another embodiment, the sequence of the region is set forth in SEQ ID No: 2 or a fragment thereof. In another embodiment, the region is homologous to SEQ ID No: 2 or a fragment thereof. In another embodiment, the sequence of the fragment comprises SEQ ID No: 5. Each possibility represents a separate embodiment of the present invention.

In another embodiment, a composition of the present invention reduces an intracellular amount of a KSHV fragment of the present invention by sequence-specific genomic targeting. In another embodiment, the composition reduces accessibility of the fragment by hybridizing to it. In another embodiment, the composition reduces accessibility of the fragment by sterically hindering it. Each possibility represents a separate embodiment of the present invention.

In another embodiment, the present invention provides a use of a composition that inhibits an initiation of DNA replication from a region of a genome of the gammaherpesvirus, in the manufacture of a medicament for reducing an incidence of a KSHV-associated KS, PEL, or multicentric Castleman's disease in a subject, wherein the composition inhibits an initiation of DNA replication from a region of a genome of the gammaherpesvirus. In another embodiment, the sequence of the region is set forth in SEQ ID No: 2 or a fragment thereof. In another embodiment, the region is homologous to SEQ ID No: 2 or a fragment thereof. In another embodiment, the sequence of the fragment comprises SEQ ID No: 5. Each possibility represents a separate embodiment of the present invention.

In another embodiment, the present invention provides a use of a composition that inhibits initiation of DNA replication from a KSHV genomic fragment of the present invention, in the manufacture of a medicament for delivering a recombinant protein to a subject, a composition comprising a DNA molecule, the DNA molecule comprising a region having a sequence set forth in SEQ ID No: 2 or a fragment thereof, and further comprising a recombinant gene encoding the recombinant protein. In another embodiment, the region is homologous to SEQ ID No: 2 or a fragment thereof. In another embodiment, the sequence of the fragment comprises SEQ ID No: 5. In another embodiment, the sequence of the region consists of SEQ ID No: 5. Each possibility represents a separate embodiment of the present invention.

In another embodiment, the present invention provides a use of a composition that inhibits initiation of DNA replication from a KSHV genomic fragment of the present invention, in the manufacture of a medicament for delivering a therapeutic RNA molecule to a subject, a composition comprising a DNA molecule, the DNA molecule comprising a region having a sequence set forth in SEQ ID No: 2 or a fragment thereof, and further comprising a recombinant gene encoding the therapeutic RNA molecule. In another embodiment, the region is homologous to SEQ ID No: 2 or a fragment thereof. In another embodiment, the sequence of the fragment comprises SEQ ID No: 5. In another embodiment, the sequence of the region consists of SEQ ID No: 5. Each possibility represents a separate embodiment of the present invention.

In another embodiment, the present invention provides a method of producing a gene therapy vector, comprising the steps of (a) obtaining a nucleotide molecule that encodes a protein of interest; and (b) inserting or subcloning into the nucleotide molecule a region of a KSHV genome of the present invention. In another embodiment, the sequence of the region is set forth in SEQ ID No: 2. In another embodiment, the sequence of the region is a fragment of SEQ ID No: 2. In another embodiment, the region is homologous to SEQ ID No: 2. In another embodiment, the region is homologous to a fragment of SEQ ID No: 2. In another embodiment, the sequence of the region consists of SEQ ID No: 2. In another embodiment, the sequence of the region comprises SEQ ID No: 5. In another embodiment, the sequence of the region consists of SEQ ID No: 5. In another embodiment, the gene therapy vector is capable of episomal replication in a eukaryotic cell. In another embodiment, the gene therapy vector is a non-integrating vector. In another embodiment, the protein of interest is a therapeutic protein. Each possibility represents a separate embodiment of the present invention.

In another embodiment, the present invention provides a method of producing a gene therapy vector, comprising the steps of (a) obtaining a nucleotide molecule that encodes a therapeutic RNA molecule; and (b) inserting or subcloning into the nucleotide molecule a region of a KSHV genome of the present invention. In another embodiment, the sequence of the region is set forth in SEQ ID No: 2. In another embodiment, the sequence of the region is a fragment of SEQ ID No: 2. In another embodiment, the region is homologous to SEQ ID No: 2. In another embodiment, the region is homologous to a fragment of SEQ ID No: 2. In another embodiment, the sequence of the region consists of SEQ ID No: 2. In another embodiment, the sequence of the region comprises SEQ ID No: 5. In another embodiment, the sequence of the region consists of SEQ ID No: 5. In another embodiment, the gene therapy vector is capable of episomal replication in a eukaryotic cell. In another embodiment, the gene therapy vector is a non-integrating vector. In another embodiment, the protein of interest is a therapeutic protein. Each possibility represents a separate embodiment of the present invention.

In another embodiment, the present invention provides a kit for studying eukaryotic DNA replication, comprising an isolated DNA molecule of the present invention and an instructional material. In another embodiment, the kit is used for studying initiation of replication. In another embodiment, the kit further comprises a means of measuring replication of the isolated DNA molecule. In another embodiment, the means is a means exemplified herein. In another embodiment, the means is any other means known in the art of measuring DNA replication. Each possibility represents a separate embodiment of the present invention.

As provided herein, a LANA-independent functional origin of replication in the KSHV genome has been identified (Examples). Human replication machinery accumulates at this origin of replication the AT region on KSHV chromatin in vivo. (Examples 6-8). Thus, DNA constructs containing this origin of replication can be used to study eukaryotic DNA replication, using techniques well known in the art.

In another embodiment, the present invention provides a method of inhibiting the development of a cancer in a subject, comprising the step of contacting the subject with a composition of the present invention. In another embodiment, the present invention provides a method of suppressing the formation of a tumor in a subject, comprising the step of contacting the subject with a composition of the present invention. In another embodiment, the present invention provides a method of inhibiting the development of a cancer in a subject, comprising the step of performing a method of the present invention. In another embodiment, the present invention provides a method of suppressing the formation of a tumor in a subject, comprising the step of performing a method of the present invention. In another embodiment, the tumor is associated with an oncogenic virus. In another embodiment, the subject is a human. Each possibility represents a separate embodiment of the present invention.

In another embodiment, the present invention provides a use of a composition that inhibits initiation of DNA replication from a KSHV genomic fragment of the present invention, in the preparation of a medicament for inhibiting the development of a cancer in a subject. In another embodiment, the present invention provides a use of a composition of the present invention in the preparation of a medicament for suppressing the formation of a tumor in a subject. In another embodiment, the tumor is associated with an oncogenic virus. In another embodiment, the subject is a human. Each possibility represents a separate embodiment of the present invention.

In another embodiment, the present invention provides a kit comprising a reagent utilized in performing a method of the present invention. In another embodiment, the present invention provides a kit comprising a composition, tool, or instrument of the present invention.

In another embodiment, a treatment protocol of the present invention is therapeutic. In another embodiment, the protocol is prophylactic. Each possibility represents a separate embodiment of the present invention.

The terms “homology,” “homologous,” etc, when in reference to any protein or peptide, refer, in another embodiment, to a percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Methods and computer programs for the alignment are well known in the art.

In another embodiment, the term “homology,” when in reference to any nucleic acid sequence, similarly indicates a percentage of nucleotides in a candidate sequence that are identical with the nucleotides of a corresponding native nucleic acid sequence.

Homology is, in another embodiment, determined by computer algorithm for sequence alignment, by methods well described in the art. In another embodiment, computer algorithm analysis of nucleic acid sequence homology includes the utilization of any number of software packages available, such as, for example, the BLAST, DOMAIN, BEAUTY (BLAST Enhanced Alignment Utility), GENPEPT and TREMBL packages.

In another embodiment, “homology” refers to identity to a sequence selected from SEQ ID No: 1-5 or 16 of greater than 70%. In another embodiment, “homology” refers to identity to a sequence selected from SEQ ID No: 1-5 or 16 of greater than 72%. In another embodiment, “homology” refers to identity to 1 of SEQ ID No: 1-5 or 16 of greater than 75%. In another embodiment, “homology” refers to identity to a sequence selected from SEQ ID No: 1-5 or 16 of greater than 78%. In another embodiment, “homology” refers to identity to 1 of SEQ ID No: 1-5 or 16 of greater than 80%. In another embodiment, “homology” refers to identity to 1 of SEQ ID No: 1-5 or 16 of greater than 82%. In another embodiment, “homology” refers to identity to a sequence selected from SEQ ID No: 1-5 or 16 of greater than 83%. In another embodiment, “homology” refers to identity to 1 of SEQ ID No: 1-5 or 16 of greater than 85%. In another embodiment, “homology” refers to identity to 1 of SEQ ID No: 1-5 or 16 of greater than 87%. In another embodiment, “homology” refers to identity to a sequence selected from SEQ ID No: 1-5 or 16 of greater than 88%. In another embodiment, “homology” refers to identity to 1 of SEQ ID No: 1-5 or 16 of greater than 90%. In another embodiment, “homology” refers to identity to 1 of SEQ ID No: 1-5 or 16 of greater than 92%. In another embodiment, “homology” refers to identity to a sequence selected from SEQ ID No: 1-5 or 16 of greater than 93%. In another embodiment, “homology” refers to identity to 1 of SEQ ID No: 1-5 or 16 of greater than 95%. In another embodiment, “homology” refers to identity to a sequence selected from SEQ ID No: 1-5 or 16 of greater than 96%. In another embodiment, “homology” refers to identity to 1 of SEQ ID No: 1-5 or 16 of greater than 97%. In another embodiment, “homology” refers to identity to 1 of SEQ ID No: 1-5 or 16 of greater than 98%. In another embodiment, “homology” refers to identity to 1 of SEQ ID No: 1-5 or 16 of greater than 99%. In another embodiment, “homology” refers to identity to 1 of SEQ ID No: 1-5 or 16 of 100%. Each possibility represents a separate embodiment of the present invention.

In another embodiment, homology is determined is via determination of candidate sequence hybridization, methods of which are well described in the art (See, for example, “Nucleic Acid Hybridization” Hames, B. D., and Higgins S. J., Eds. (1985); Sambrook et al., 2001, Molecular Cloning, A Laboratory Manual, Cold Spring Harbor Press, N.Y.; and Ausubel et al., 1989, Current Protocols in Molecular Biology, Green Publishing Associates and Wiley Interscience, N.Y). In another embodiment, methods of hybridization are carried out under moderate to stringent conditions, to the complement of a DNA encoding a native caspase peptide. Hybridization conditions being, for example, overnight incubation at 42° C. in a solution comprising: 10-20% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared salmon sperm DNA.

In another embodiment of the present invention, “nucleic acids” refers to a string of at least two base-sugar-phosphate combinations. The term includes, in another embodiment, DNA and RNA. “Nucleotides” refers, in another embodiment, to the monomeric units of nucleic acid polymers. RNA is, in another embodiment, in the form of a tRNA (transfer RNA), snRNA (small nuclear RNA), rRNA (ribosomal RNA), mRNA (messenger RNA), anti-sense RNA, small inhibitory RNA (siRNA), micro RNA (miRNA) and ribozymes. The use of siRNA and miRNA has been described (Caudy A A et al, Genes & Devel 16: 2491-96 and references cited therein). In another embodiment, DNA is in form of plasmid DNA, viral DNA, linear DNA, or chromosomal DNA or derivatives of these groups. In another embodiment, these forms of DNA and RNA are single, double, triple, or quadruple stranded. The term also includes, in another embodiment, artificial nucleic acids that contain other types of backbones but the same bases. In another embodiment, the artificial nucleic acid is a PNA (peptide nucleic acid). PNA contain peptide backbones and nucleotide bases and are able to bind, in another embodiment, to both DNA and RNA molecules. In another embodiment, the nucleotide is oxetane modified. In another embodiment, the nucleotide is modified by replacement of one or more phosphodiester bonds with a phosphorothioate bond. In another embodiment, the artificial nucleic acid contains any other variant of the phosphate backbone of native nucleic acids known in the art. The use of phosphothiorate nucleic acids and PNA are known to those skilled in the art, and are described in, for example, Neilsen P E, Curr Opin Struct Biol 9:353-57; and Raz N K et al, Biochem Biophys Res Commun 297: 1075-84. The production and use of nucleic acids is known to those skilled in art and is described, for example, in Molecular Cloning, (2001), Sambrook and Russell, eds. and Methods in Enzymology: Methods for molecular cloning in eukaryotic cells (2003) Purchio and G. C. Fareed. Each nucleic acid derivative represents a separate embodiment of the present invention.

Protein and/or peptide homology for any amino acid sequence listed herein is determined, in another embodiment, by methods well described in the art, including immunoblot analysis, or via computer algorithm analysis of amino acid sequences, utilizing any of a number of software packages available, via established methods. Some of these packages include the FASTA, BLAST, MPsrch or Scanps packages, and can employ the use of the Smith and Waterman algorithms, and/or global/local or BLOCKS alignments for analysis, for example. Each method of determining homology represents a separate embodiment of the present invention.

In another embodiment, the phrase “contacting a cell” or “contacting a population” refers to a method of exposure that can be direct or indirect. In another embodiment, such contact comprises direct injection of the cell through any means well known in the art, such as microinjection. In another embodiment, supply to the cell is indirect, such as via provision in a culture medium that surrounds the cell, or administration to a subject, or via any other route known in the art. In another embodiment, the term “contacting” means that the composition of the present invention is introduced into a subject receiving treatment. Each possibility represents a separate embodiment of the present invention.

### Pharmaceutical Compositions

“Pharmaceutical composition” refers, in another embodiment, to a therapeutically effective amount of the active ingredient, together with a pharmaceutically acceptable carrier or diluent. A “therapeutically effective amount” refers, in another embodiment, to an amount that provides a therapeutic effect for a given condition and administration regimen.

The pharmaceutical compositions containing the active ingredient can be, in another embodiment, administered to a subject by any method known to a person skilled in the art, such as parenterally, paracancerally, transmucosally, transdermally, intramuscularly, intravenously, intra-dermally, subcutaneously, intra-peritonealy, intra-ventricularly, intra-cranially, intra-vaginally or intra-tumorally.

In another embodiment of methods and compositions of the present invention, the pharmaceutical compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In another embodiment of the present invention, the active ingredient is formulated in a capsule. In accordance with this embodiment, the compositions of the present invention comprise, in addition to the active compound and the inert carrier or diluent, a hard gelating capsule.

In another embodiment, the pharmaceutical compositions are administered by intravenous, intra-arterial, or intra-muscular injection of a liquid preparation. Suitable liquid formulations include solutions, suspensions, dispersions, emulsions, oils and the like. In another embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intramuscularly and are thus formulated in a form suitable for intra-muscular administration.

In another embodiment, the pharmaceutical compositions are administered topically to body surfaces and are thus formulated in a form suitable for topical administration. Suitable topical formulations include gels, ointments, creams, lotions, drops and the like.

In another embodiment, the pharmaceutical composition is administered as a suppository, for example a rectal suppository or a urethral suppository. In another embodiment, the pharmaceutical composition is administered by subcutaneous implantation of a pellet. In another embodiment, the pellet provides for controlled release of active ingredient agent over a period of time.

In another embodiment, the active compound is delivered in a vesicle, e.g. a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid).

As used herein “pharmaceutically acceptable carriers or diluents” are well known to those skilled in the art. The carrier or diluent can be, in various embodiments, a solid carrier or diluent for solid formulations, a liquid carrier or diluent for liquid formulations, or mixtures thereof.

In another embodiment, solid carriers/diluents include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

In other embodiments, pharmaceutically acceptable carriers for liquid formulations can be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil.

Parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of petroleum, animal, vegetable, or synthetic origin, for example, peanut oil, soybean oil, mineral oil, olive oil, sunflower oil, and fish-liver oil.

In another embodiment, the compositions further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCI., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents (e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants. Each of the above excipients represents a separate embodiment of the present invention.

In another embodiment, the pharmaceutical compositions utilized in methods of the present invention are controlled-release compositions, i.e. compositions in which the active ingredient is released over a period of time after administration. Controlled- or sustained-release compositions include formulation in lipophilic depots (e.g. fatty acids, waxes, oils). In another embodiment, the composition is an immediate-release composition, i.e. a composition wherein all of the active ingredient compound is released immediately after administration.

In another embodiment, the pharmaceutical composition is delivered by a controlled release system. In another embodiment, the agent can be administered using intravenous infusion, an implantable osmotic pump, a transdermal patch, liposomes, or other modes of administration. In another embodiment, a pump is used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials are used; e.g. in microspheres in or an implant. In yet another embodiment, a controlled release system is placed in proximity to the therapeutic target, thus requiring only a fraction of the systemic dose (see, e.g., Goodson, in Medical Applications of Controlled Release, supra, vol. 2, pp. 115-138 (1984); and Langer R, Science 249: 1527-1533 (1990).

The compositions also include, in another embodiment, incorporation of the active material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance.

Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

Also comprehended by the invention are compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds exhibit, in another embodiment, substantially longer half-lives in blood following intravenous injection than do the corresponding unmodified compounds. In another embodiment, such modifications also increase the compound's solubility in aqueous solution, eliminate aggregation, enhance the physical and chemical stability of the compound, and greatly reduce the immunogenicity and reactivity of the compound. In another embodiment, the desired in vivo biological activity is achieved by the administration of such polymer-compound abducts less frequently or in lower doses than with the unmodified compound.

The preparation of pharmaceutical compositions that contain an active component, for example by mixing, granulating, or tablet-forming processes, is well known in the art. The active therapeutic ingredient is mixed, in another embodiment, with excipients that are pharmaceutically acceptable and compatible with the active ingredient. For oral administration, the active ingredient is mixed, in another embodiment, with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. For parenteral administration, the active ingredient agents is converted, in another embodiment, into a solution, suspension, or emulsion, if desired with the substances customary and suitable for this purpose, for example, solubilizers or other substances.

An active component is, in another embodiment, formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Each of the above additives, excipients, formulations and methods of administration represents a separate embodiment of the present invention.

### EXPERIMENTAL DETAILS SECTION

### Example 1

### Fragment G of the LUR of the KSHV Genome Supports Lana-Independent Replication

### Materials and Experimental Methods

### Subcloning of Z6 Fragments

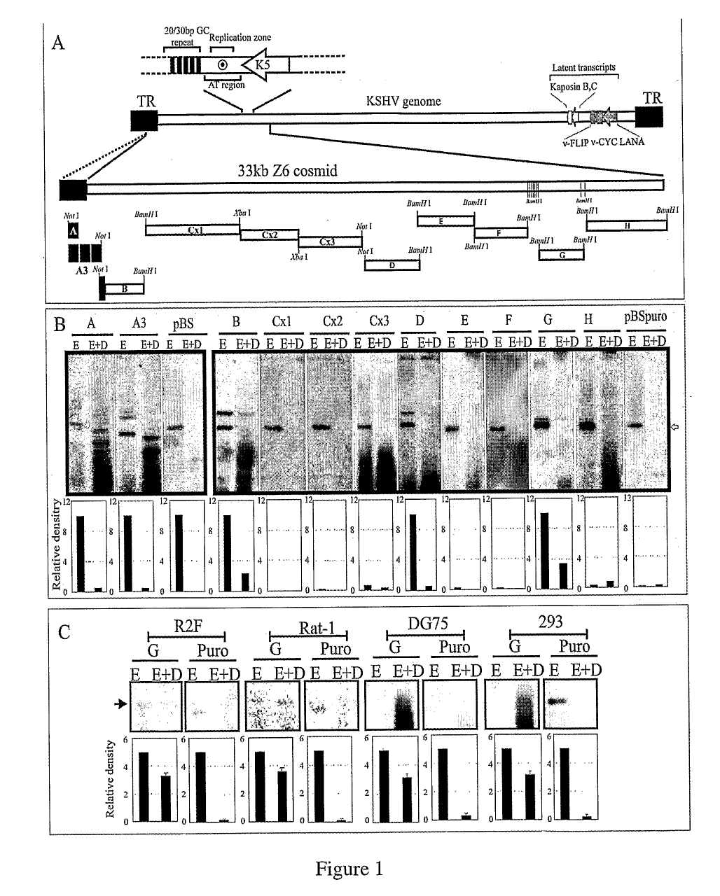

pBSpuro, which lacks a eukaryotic origin of replication, was constructed by subcloning the puromycin resistance expression cassette from pBABEpuro to the Sal I and Cla I sites of the pBluescript (Stratagene) multiple cloning site (MCS). Z6 cosmid was digested with BamHI, NotI and XbaI, which generated the fragments shown in FIG. 1A. Each fragment was cloned with respective enzyme sites of pBSpuro. Clones containing single and 3 copies of terminal repeats were termed pBSpuroA and pBSpuroA3, respectively. pBSpuroB clone comprised of the extreme left end of the genome had the K1 ORF as well as a region of the TR. C fragment (14.8 kb), obtained by BamHI and NotI was further digested with XbaI to yield three fragments (C×1, C×2 and C×3) which were cloned with respective enzymes. The D, E and F fragments were cloned with respective enzyme and referred to as pBSpuro D, E, F, G and H. Each clone was confirmed by restriction digestion and sequence analysis. pBSpuroTR was used as a control for replication and incorporation of BrdU.

### Short-Term Replication Assays

pBSpuro clones A-H, containing various regions of Z6 cosmid, were transfected into HEK293 cells using Gene Pulser® (Bio-Rad). 10 million cells were transfected with 25 μg of respective DNA with or without LANA expression vector. Puromycin (1 μg/ml) was added to the medium for selection of transfected cells. 96 h post-transfection plasmid DNA was extracted using modified Hirt's procedure (described below), then digested with EcoRI (to linearize) or EcoRI+DpnI. DNA were electrophoresed on a 0.6% agarose gel, transferred to a nylon membrane, hybridized with ³²P-labeled pBSpuro probe, and signals were detected using a PhosphorImager (Molecular Dynamics Inc.). Signals were quantified using Image Quant (Molecular dynamics). pBluescript (pBS) DNA was spiked into the digestion reaction to test the completion of digestion.

### Hirt's Extraction

Medium from the 100 mm plates was removed and cells were washed with PBS followed by lysing the cells in plates with a 1:2 mixture of Solution I and II (Solution I: Tris, Glucose, and EDTA; Solution II: SDS and NaOH). Lysed cells were transferred to a tube and added with potassium acetate (Solution III). The lysate was incubated on ice for 10 min, followed by centrifugation at 8,000 rpm for 10 min. The supernatant was further extracted with phenol using Phenol:Chloroform:IAA, and DNA was precipitated using 0.6 volume of 2-propanol. The pellet was dried and dissolved in TE with RNAase and incubated at 37° C. for 30 min followed by Proteinase K treatment. Samples were extracted a second time with phenol and dissolved in water after precipitation.

### Results

In order to identify cis-acting elements of the KSHV genome that are important for DNA maintenance and replication, portions of the Z6 restriction fragment from the left end of the KSHV long unique region (LUR) were analyzed for ability to support replication in a short-term assay. Cloning of Z6 DNA fragments into pBSpuro, a vector lacking a eukaryotic origin of replication, allowed straightforward screening of candidate viral cis-acting elements capable of replication. In this assay, only inserts containing a KSHV origin of replication complement the absence of an origin in the vector and allow for efficient, long-term maintenance of the puromycin resistance gene in the transfected cells.

In order to differentiate between input E. coli-replicated plasmids and plasmids replicated in the transfected HEK293 cells lacking Dam methylases, Hirt DNA was subjected to Dpn I digestion before electrophoresis and Southern analysis. Efficiency of replication was determined by the ratio of DpnI-resistant copies verses input plasmid copies. Fragments of the Z6 cosmid cloned into the pBSpuro vector exhibited various degree of replication (FIG. 1B). As expected, pBSpuro containing a single copy (A) or 3 copies (A3) of TR replicated in LANA expressing cells, but not in the absence of LANA. The pBSpuroB plasmid, containing 1.8 kilobase pairs (kbp) from the left end of the KSHV genome as well as a 600 bp region of the TR (B), replicated as efficiently as pBSpuroA/A3; this is due to the presence of the TR fragment. pBSpuro plasmids containing other fragments of LUR, C×3, D and G, replicated as well to varying degrees, with fragment G exhibiting the greatest replication efficiency. Replication efficiency of fragment G was not reduced in the absence of LANA expression, indicating that fragment G contains a LANA-independent origin of replication (Table 1). By contrast, replication of plasmids containing A, A3, B, C×3, and D was reduced in the absence of LANA expression.

TABLE 1
Comparison of the DpnI resistant bands 96 h post-transfection.

| Fragments | Ratio |
|---|---|
| A | 2.3 |
| A3 | 0.3 |
| B | 1.8 |
| Cx3 | 0 |
| D | 0.58 |
| G | 4.8 |

Relative amounts were calculated based on the intensity of the DpnI resistant DNA signals. The signals were calculated as a percentage based on the input DNA control which was 10% of the total used for DpnI digestion.

The sequence of fragment G is as follows.

(SEQ ID No: 1)

ggatcccggcgcgccaccctccccggcaacaacctgttgccatgtatggc
gatttgtatcagtcacaagcacacaacccctgctagtattaatggtgttt
aaaacgttctacacgtacggcggaccgcatccgtcgcaagcacgcgcata
taacccccaaatgcaccatgatgagaagcacagccacgcgtcaaaaaact
ttaaaaacatcgttatccaatatcattaaaaaccacaccgaaatttacac
aggtagcacgtcaccgtgttagtgtcacccactgtacacaaggcgtgtcg
tatatgtagtataggtatttgatgaggcggaagcatatcccgcttccagc
gaacggaaataagaatcatccgttccagcatttattcaaagagggcacag
aggattcacattgtttagagagagtttttcttagtcaccattccatactt
gggcagtattggcctacgatttgggcgacgtttcaggctggtctattctc
cgtccacttttccccggctattctgtcccagcataggctcttgaaataaa
caatgtttaccgagtaaaaggttccactcaccctcatttgtcgttgcacc
catcccccctttgcttaatcacccgaaaactagaggacacggatggaaaa
catatcgcacgcgggttgtttgaaagtcaacagctacttgtttttaatga
ggacagatttgggcacaggccagagggtaaagccctacgtgtgcgcgggg
gggggggtgtatacgctgcgaaaacctgcacggtgcataacacccagggc
gtcacgtcacatatctctgtgcacccaagtggttgttcaaccgttgtttt
ttggatgatttttccgcaccggcttttttgtgggcgcgcataggtcggta
cgcgctgtccccctaagtcccgcacggtcgttcgggcccccgtccggctc
gtctccggatgaaccgtcacgttctttgtctccagaggcgacgtctcctt
cagatgactcgtccgtgggctcctcgtccgtcccgcccgcgggtccgaca
aggaccgtcaattcgatgttatcttcgttcgcggttggccggcgcggccg
tcggtatggcagtacggtcacccgggtgttatttgccgcgtataatgccc
tcacagtgccacttacgcggcatatgccgccaaatgcaaacacaataaat
atttggtaaaacccaaagaagcagagaaaaccgagcacggccccggggga
gaatgttcccgcaggagcagttaggatgaccaggagcgtccaggtgcaca
acgccacgccgacaagcccagccaccaccacagacatcagcagaaacagt
tcaaaaatttcttggcgctccatctccggccacaggttaaggcgactacg
ccactgcgtgcgcgtgcggtatataacgcgacacatttgacaggccgtgt
ttcgagacactgttagccaagtgcttaaacactgcgggtggacgacatcc
agctctccggtacaggcgcaggggtgtatgccctcgttccccacctcttc
cctacatatccagcagatgggtccctctacaccctcttctacgtccttag
acgccatctctgcagctggggtggaagtctgaaaaagggaaaggggaggt
gagcagagtgcccagttagtctccgacccgccgtccgccctactgtcgct
atcccgccttgacagatgtctaacgtattcacggacgccacatgtgtgtc
tattttcctacatccaggctttccctggaaaactgtcacaacccaccctg
ctttagctctacatctgtatttttgtttacgcacaggatcaacgcttcgt
gcccgtccacccccgcgctctccgcctgtgtttggaggttttatgagtgg
ttagttctaggcagctccggacaagttgtccaaaacacggcgcg.

To test replication of the KSHV G fragment in primary cells, the G fragment-containing vector (pBSpuroG) or empty vector (pBSpuro) were transfected into human foreskin fibroblast (R2F), Rat-1 and DG75. Hirt DNA extracted 96 h post transfection and digestion with either EcoRI (E) or EcoRI and DpnI (E+D) revealed that the G fragment supported plasmid replication (FIG. 1C).

These findings show that fragment G of the KSHV genome contains a functional origin of replication. These results also show that binding of ORCs and MCMs is specific to particular DNA elements of the KSHV genome.

### Example 2

### Further Evidence that Fragment Supports Lana-Independent Replication

### Materials and Experimental Methods

### Long-Term Selection and Gardella Gel Analysis

10 μg of pBSpuroG was transfected into 5×10̂6 293 cells, which were plated onto 100 millimeter (mm) plates containing 1.0 microgram (mcg)/ml puromycin and incubated for 3 weeks to select for transfected cells. Colonies were further sub-cloned and analyzed by Gardella gels (Gardella, T, Medveczky, P, et al, 1984, J Virol 50: 248-254). 2×10̂6 cells from 3 colonies were washed with cold phosphate-buffered saline and resuspended in loading buffer (10% Ficol, 1×TBE and RNAase). A lysing agarose plug (containing 1% SDS and 1 mg/ml Pronase E in 0.8% agarose) was poured, then cells were immediately loaded and resolved for 4 h at 20 volts (V), then at 80 V for 14 hr. DNA was transferred to Gene Screen® (NEN) membrane and subjected for detection by Southern blot using ³²P-labeled pBSpuro probe.

### Preparation of Chromosome Spread and In-Situ Detection of Replicated Plasmid

Colonies selected with pBSpuroG and 293 cells were arrested in mitosis by treatment with colchicines, then washed with phosphate-buffered saline, detached from the flask by vigorous shaking, centrifuged at 600 rpm for 5 min, resuspended gently in 75 mM KCl, and incubated at room temperature (RT) for 20 min. Cells were centrifuged at 600 rpm after adding several drops of fixative (3:1 methanol:acetic acid), supernatant was removed, cells were resuspended in chilled fixative, and the above process was repeated 3 more times. Cells were resuspended in 0.5 ml fresh fixative and dropped from a height of 2-3 feet onto a clean slide held at 45 degree angle (Verma, S C, and Robertson, E S, 2003, J Virol 77: 12494-12506). Chromosomes were aged at RT, and plasmid was detected by overnight hybridization at 37° C. to biotin-labeled pBSpuro DNA as a probe, followed by detection with a tyramide-rhodamine signal amplification system (Perkin-Elmer Life Sciences, Inc, Boston, Mass.). Chromosomes were stained with DAPI (large clumps); smaller dots, which are the site of probe hybridization, represent individual copies of plasmids.

### Separation of BrdU Labeled DNA in CsCl Density Equilibrium:

Plasmids, pBSpuroG and pBSpuro were transfected into 2×10⁷ 293 cells. 24 h post-transfection cells were pulsed with BrdU (30 μg/ml) and incubated for 24 or 48 h. Total DNA isolated from cells was sonicated, loaded onto a CsCl density gradient, and the refractive index (r.i.) was adjusted to 1.4038. Tubes were centrifuged in a Beckman SW41 rotor for 24 h at 40,000 rpm, then for another 24 h at 30,000 rpm. Gradients were fractionated into 250 μl fractions from the top, the r.i. of fractions were determined using a refractometer (Fisher Scientific), then fractions were dialyzed by drop dialysis technique using a 0.025 μM Millipore membrane filter (Millipore Inc.) and precipitated. Total DNA in each fraction was quantified using SYBR Green. Plasmid copies in fractions were calculated by quantitative PCR.

### Results

To determine whether replication mediated by G fragment is synchronous with the cellular DNA replication mechanism. 2.0×10⁷ 293 cells were transfected with pBSpuroG or pBSpuro vector and pulsed with 5-bromo-3-deoxyuridine (BrdU), an analog of thymidine, 24 h post transfection to increase the buoyant density of the replicated DNA for evaluation by CsCl equilibrium density gradient. During semi-conservative replication in the presence of density label (BrdU), the newly synthesized plasmid incorporates BrdU and forms a hybrid of light:heavy (H:L), after 1 or 2 rounds of synthesis and heavy:heavy (H:H) after 2 or more rounds of replication and can easily be distinguished from light:light (L:L) unreplicated DNA. Since 293 cells divide in approximately 24 h, cells were harvested at 24 and 48 h post BrdU pulse in order to allow sufficient time for 1 and 2 rounds of replication, respectively. DNA isolated from these cells was subjected to CsCl density gradient centrifugation and the distribution of total DNA, a measure of BrdU incorporation, on the gradient was analyzed.

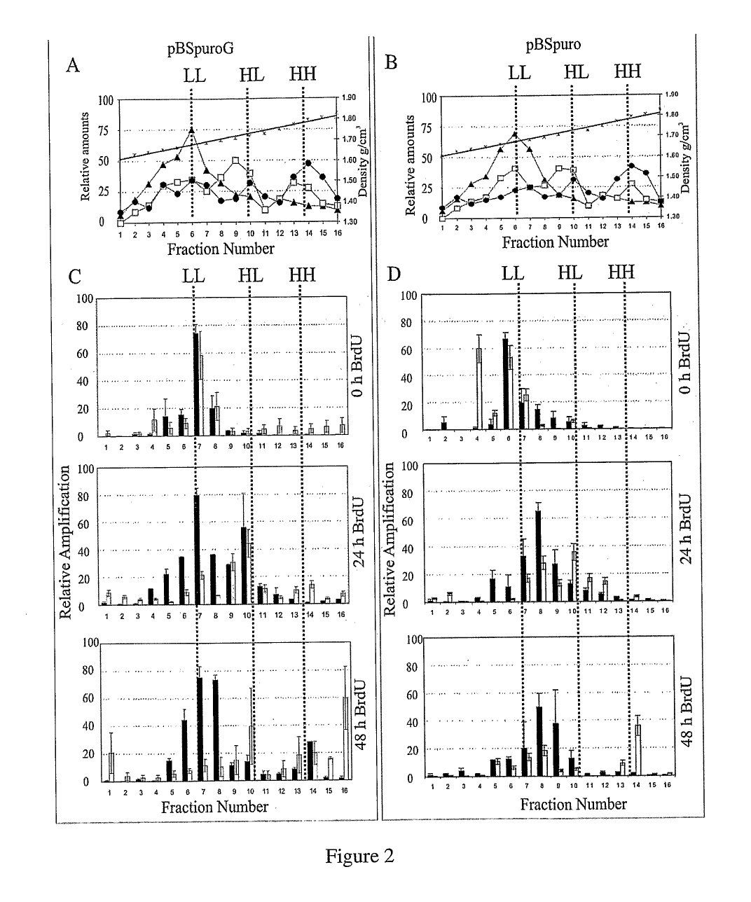

FIG. 2A-B depicts a peak at heavy-light (hemi-substituted) position within a 24-hour pulse. DNA from cells incubated with BrdU for another 24 h showed a peak at H:H along with H:L suggesting that the second round of replication substituted both the strands. The distribution of density label DNA peaks are exactly as seen in semi-conservative DNA replication. The density shift of the plasmid DNA was analyzed by quantitative PCR for the presence of pBSpuroG and pBSpuro in different fractions from CsCl gradient of DNA pulsed for 0, 24 and 48 h. Quantitation of pBSpuroG copies (expressed as relative amplification) in L:L, H:L and H:H fractions clearly showed that plasmid DNA (FIG. 2, black bars) peaks at L:L at t=0 BrdU pulsing time, similar to the cellular DNA (FIG. 2, gray bars). After 24 h, a fraction of pBSpuroG shifted to the H:L peak similar to the cellular DNA shift (FIG. 2, gray bars), which was further shifted to the H:H peak after 2 rounds of replication (FIG. 2C). Relative copies of pBSpuroG and cellular DNA in these fractions clearly demonstrate that the plasmid replicated in synchrony with cellular DNA, in a once-per-cell cycle manner. pBSpuro lacking G fragment showed a slight shift of the plasmid copies (from fraction 6 to 8/9) below that expected for a single rounds of replication, showing that empty vector did not incorporate BrdU as efficiently as the G fragment (FIG. 2D). Cellular DNA from pBSpuro transfected cells also exhibited a shift in peak from L:L to H:L and H:H, showing that the cells were actively growing in the same manner as pBSpuroG. Thus, after 48 h with 2 rounds of replication, pBSpuroG exhibited definite incorporation of BrdU in H:H fraction, whereas little or no change was observed with the corresponding fraction in pBSpuro control.

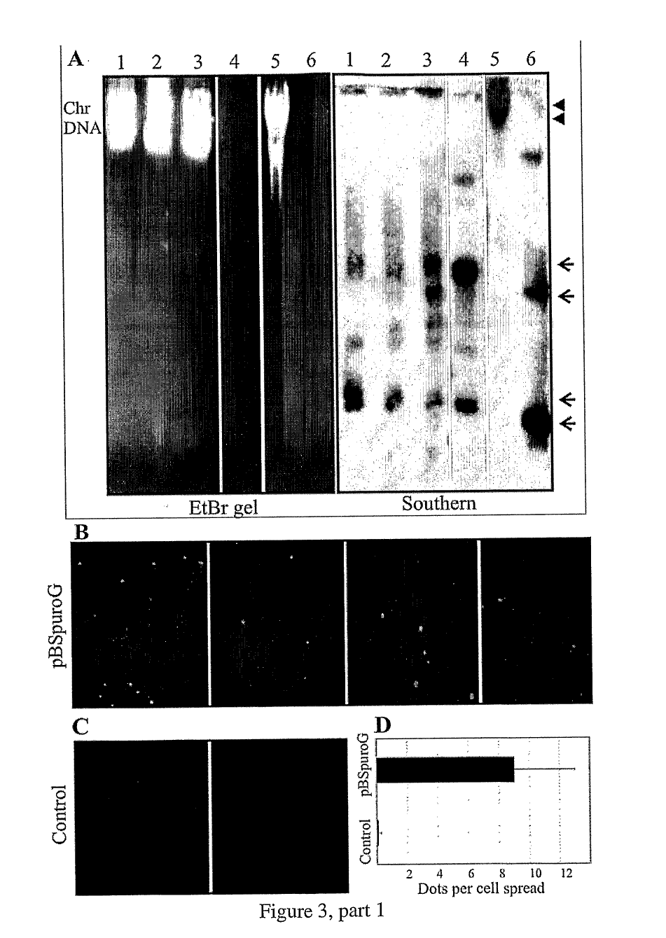

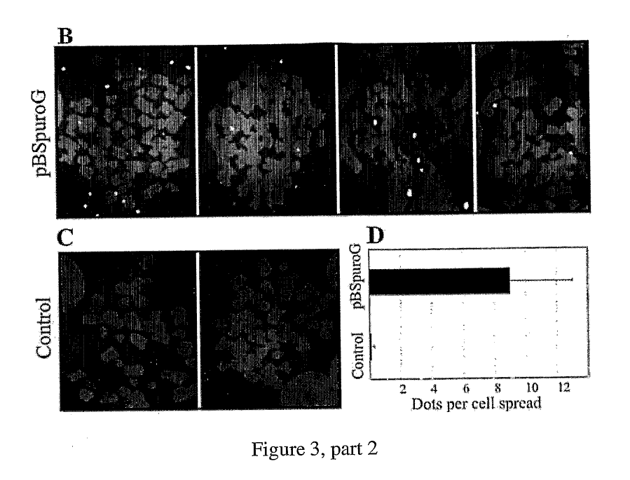

To determine whether pBSpuroG was episomally maintained, clonal populations of HEK293 transfected with pBSpuroG were subjected to Gardella gel analysis. 2.0 million cells from 3 independent colonies selected for 6 weeks were analyzed by in-situ lysis. Southern analysis detected episomal plasmid in the clonal population of pBSpuroG selected 293 cells comparable in size to bacterially replicated plasmid (FIG. 3A, lane 4). The majority of the plasmid copies detected were super-coiled, showing that these plasmids were replicated and maintained independently (FIG. 3A, lanes 1-3, arrow). Colonies selected with pBSpuro vector did not exhibit the presence of a plasmid band; rather a band of genomic size was detected, indicative of integration of the plasmid DNA into the host genome (FIG. 3, lane 5). To determine the number of plasmid copies per cell, chromosome spreads from clonal populations of 293 cells stably transfected with pBSpuroG were prepared. In-situ hybridization using biotin-labeled probe followed by detection with Streptavidin showed 6-12 copies of the plasmid per cell as an average of multiple counts per clonal line (FIG. 3D).

These findings show that pBSpuroG is not integrated into the host genome, but rather is maintained long-term as an episomal DNA element.

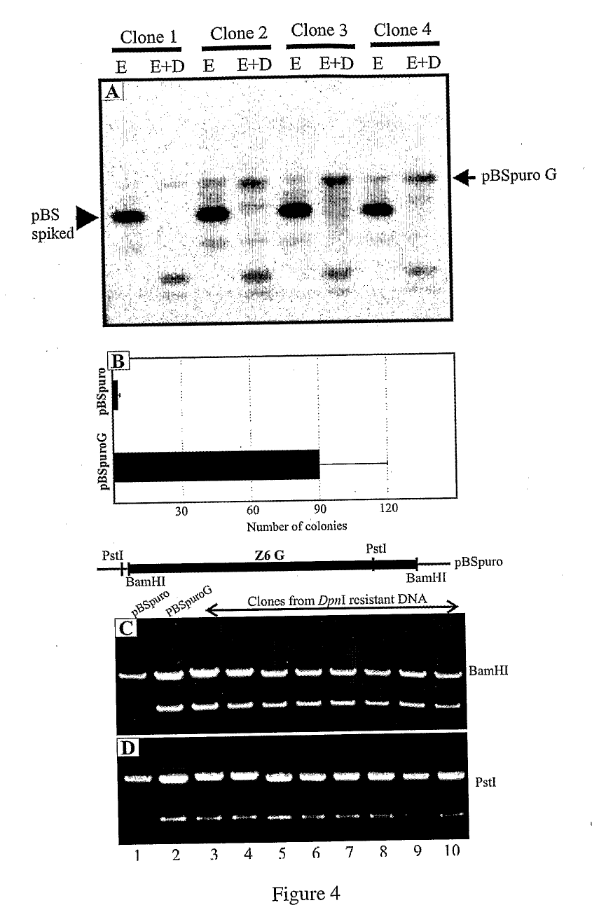

To test whether the G region contained the cis-acting origin that mediated replication, low molecular weight DNA, isolated from pBSpuroG clones selected for 4 weeks, was subjected to DpnI sensitivity analysis. DpnI-resistant copies of pBSpuroG were present in the DNA isolates (indicated by arrow) whereas the spiked control plasmid, pBS was completely digested (FIG. 4A). Thus, episomally-maintained pBSpuroG replicates during successive rounds of division of eukaryotic cells. To further confirm the contribution of the G fragment for replication, G fragment in pBluescript (lacking puromycin selection marker) was transfected into 293 cells and incubated for 96 h without selection of puromycin. The DpnI sensitivity analysis of isolated DNA without selection also showed similar replication efficiency, showing that the replication of the plasmid was not due to the selection pressure of puromycin.

Thus, long-term maintenance of pBSpuroG is due to plasmid replication with a functional cis-acting origin of replication, which is contained in the inserted G region.

### Example 3

### pBSpuroG Replicates in its Native Form and is not Rearranged During Long-Term Selection

### Materials and Experimental Methods

Analysis of Plasmid Isolated from Long-Term Selected Colonies

Low molecular weight DNA spiked with pBS plasmid (control for digestion) was extracted from pBSpuroG long term (8 weeks) selected clones as described in Example 1 and digested with either EcoRI or EcoRI+DpnI. The product was resolved on an 0.8% agarose gel and transferred to Gene Screen® membrane for detection of replicated copies using pBSpuro as a probe. 10% of the product was transformed into E. coli and selected on ampicillin plates, the number of colonies was counted and plotted, and plasmids from transformants were isolated and their restriction patterns analyzed using BamHI and PstI digestion.

### Results

To determine whether episomally maintained pBSpuroG in long-term selected cells replicated in its native form, DNA extracted from four clones were subjected for DpnI digestion. Transformation of digested DNA into E. coli yielded a significant number of ampicillin-resistant colonies (FIG. 4B). The restriction pattern of plasmids isolated from these colonies matched with control parental pBSpuroG, demonstrating that the replicated plasmid was maintained in its native form (FIG. 4C-D). As a control, digested Hirt DNA from pBSpuro cells did not confer ampicillin resistance upon E. coli, showing that the empty vector had been either integrated into the host genome or otherwise rearranged, so that the plasmids did not segregate and replicate as independent episomes.

To further confirm these results, the recovered pBSpuroG plasmid, was further analyzed for the sequence integrity by sequencing the G fragment. The sequence of the clones was identical to the parental plasmid, confirming the integrity of the replicated plasmids

These findings corroborate the above results by showing that pBSpuroG is maintained episomally in eukaryotic cells, and demonstrate that the replicated plasmids were maintained in their native state.

### Example 4

### An AT-Rich Region within the G Fragment Supports Replication

### Materials and Experimental Methods

Analysis of pBSpuroG Sequence and Short Term Replication Assay

The AT-rich region (24877-25712) and K5 ORF (25713-26483) regions of fragment G were cloned separately into same vector backbone as follows: The K5 region was removed by AccI+EcoRI digestion to create the AT vector. The AT region was removed by NotI+AccI digestion to create the K5V vector. Products where then blunt-end re-ligated. Constructs were subjected to short term replication assay in 293 cells, as described in Example 1. pBS empty vector was used as a control for transfection and replication. Sequences of the G fragment and AT rich and K5 fragments are set forth below:

AT-rich region:

(SEQ ID No: 2)

Ggatcccggcgcgccaccctccccggcaacaacctgttgccatgtatggc
gatttgtatcagtcacaagcacacaacccctgctagtattaatggtgttt
aaaacgttctacacgtacggcggaccgcatccgtcgcaagcacgcgcata
taacccccaaatgcaccatgatgagaagcacagccacgcgtcaaaaaact
ttaaaaacatcgttatccaatatcattaaaaaccacaccgaaatttacac
aggtagcacgtcaccgtgttagtgtcacccactgtacacaaggcgtgtcg
tatatgtagtataggtatttgatgaggcggaagcatatcccgcttccagc
gaacggaaataagaatcatccgttccagcatttattcaaagagggcacag
aggattcacattgtttagagagagtttttcttagtcaccattccatactt
gggcagtattggcctacgatttgggcgacgtttcaggctggtctattctc
cgtccacttttccccggctattctgtcccagcataggctcttgaaataaa
caatgtttaccgagtaaaaggttccactcaccctcatttgtcgttgcacc
catcccccctttgcttaatcacccgaaaactagaggacacggatggaaaa
catatcgcacgcgggttgtttgaaagtcaacagctacttgtttttaatga
ggacagatttgggcacaggccagagggtaaagccctacgtgtgcgcgggg
gggggggtgtatacgctgcgaaaacctgcacggtgcataacacccagggc
gtcacgtcacatatctctgtgcacccaagtggttgt.

K5 fragment:

(SEQ ID No: 3)

tcaaccgttgttttttggatgatttttccgcaccggcttttttgtgggcg
cgcataggtcggtacgcgctgtccccctaagtcccgcacggtcgttcggg
cccccgtccggctcgtctccggatgaaccgtcacgttctttgtctccaga
ggcgacgtctccttcagatgactcgtccgtgggctcctcgtccgtcccgc
ccgcgggtccgacaaggaccgtcaattcgatgttatcttcgttcgcggtt
ggccggcgcggccgtcggtatggcagtacggtcacccgggtgttatttgc
cgcgtataatgccctcacagtgccacttacgcggcatatgccgccaaatg
caaacacaataaatatttggtaaaacccaaagaagcagagaaaaccgagc
acggccccgggggagaatgttcccgcaggagcagttaggatgaccaggag
cgtccaggtgcacaacgccacgccgacaagcccagccaccaccacagaca
tcagcagaaacagttcaaaaatttcttggcgctccatctccggccacagg
ttaaggcgactacgccactgcgtgcgcgtgcggtatataacgcgacacat
ttgacaggccgtgtttcgagacactgttagccaagtgcttaaacactgcg
ggtggacgacatccagctctccggtacaggcgcaggggtgtatgccctcg
ttccccacctcttccctacatatccagcagatgggtccctctacaccctc
ttctacgtccttagacgccat.

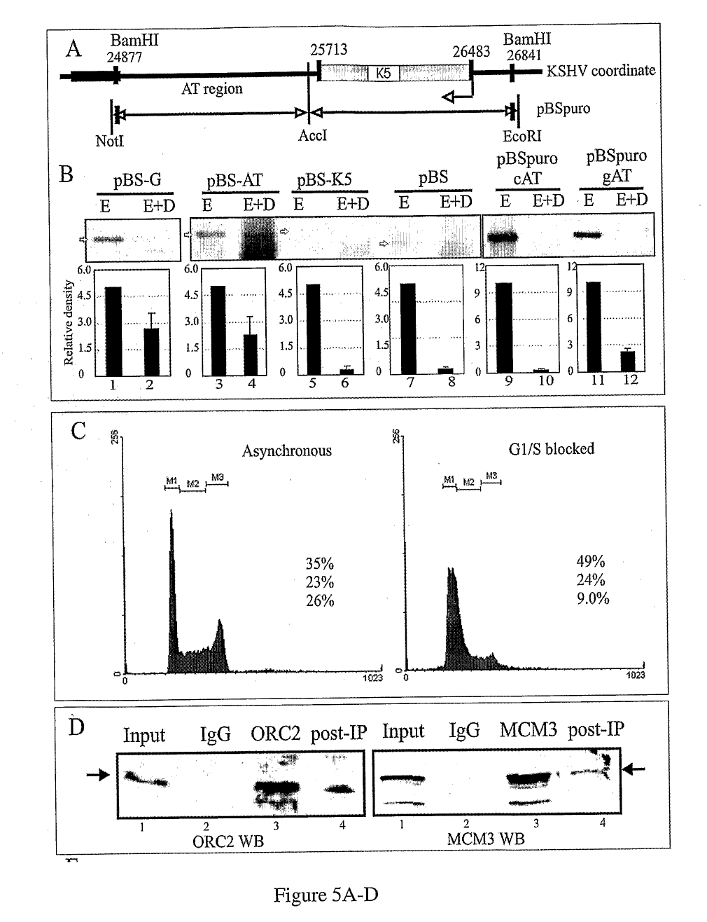

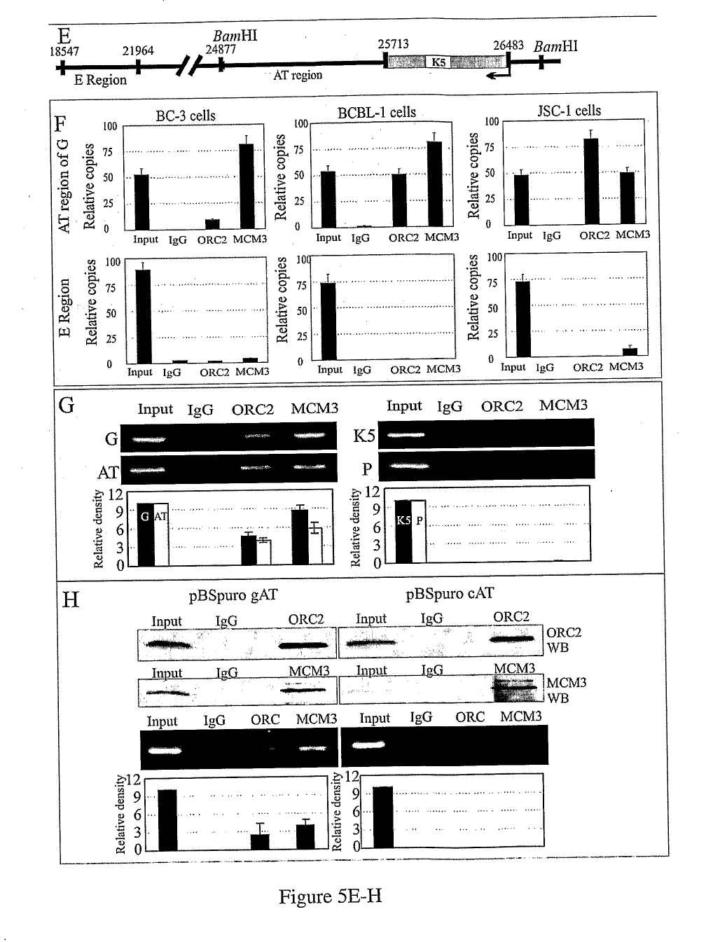

Scanning analysis of GC content is depicted in FIG. 5I.

### Contol AT Rich Sequence

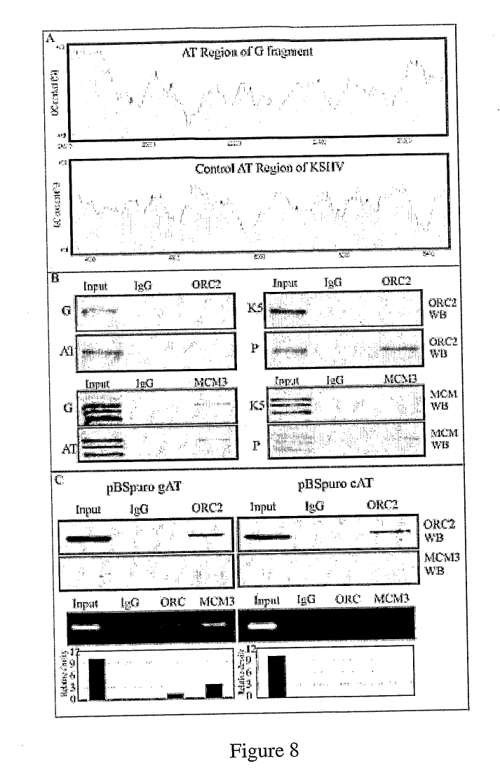

The C×1 region of Z6, which did not replicate during the short term replication assay (FIG. 1B), was selected for identification of a region with 52% AT content. The C×1 fragment sequence was analyzed using Vector NTI software (Invitrogen Inc. Carlsbad, Calif.) (FIG. 8A). The control AT rich region was amplified using specific primers: (S con AT 5′-aatggatccAGAGACCCATATGTGATCACG-3′; SEQ ID No: 19, AS-con AT 5′-aatggatccTCGACTCTGCTCCGGTGG-3′; SEQ ID No: 20) containing BamHI sites on both sides of the amplicon, then was cloned at BamHI site of the pBSpuro vector, generating pBSpuro cAT.

### Results

Sequence analysis of the G fragment revealed the presence of a non-coding AT-rich region adjacent to the coding sequence for ORF K5 (referred to herein as the “AT region”; FIG. 5A). To test the role of each individual region, the non-coding AT-rich sequence and K5 region were cloned separately into pBluescript (pBS), generating pBS-AT and pBS-K5, and the replicative ability of these fragments was tested as described in Example 1. A DpnI-resistant band was present in cells transfected with the AT region-containing plasmid, but not the K5-containing plasmid (FIG. 5B, lanes 4 and 6). The same results were observed in multiple experiments. pBS-AT and pBSpuroG exhibited similar efficiencies of replication, showing that the AT region is predominantly responsible for the replication activity and can function to initiate replication.

To confirm that the AT region of G fragment specifically supports replication, an identical-sized region of the KSHV genome with similar AT content (˜52%) and length (760 bp) was cloned into pBSpuro (“pBSpuro cAT”) and replication efficiency compared with plasmid containing the AT region of the G fragment (pBSpuro gAT). DpnI analysis of the Hirt DNA extracted 96 h post transfection revealed the absence of replicated plasmids in control AT-containing plasmids in contrast to the AT region of G fragment (FIG. 5B). This suggests that the DNA sequence present in AT region of the G fragment but not the percent AT content is important for DNA replication.

### Example 5

### The AT Region Contains an Autonomous Replicating Sequence Consensus Site (ACS)

### Materials and Experimental Methods

The AT region nucleotide sequence was aligned with the ACS (Autonomous Replicating Sequence; ARS) consensus sequence of S. cerevisiae using multiple sequence alignment program (Corpet F, Multiple sequence alignment with hierarchical clustering. Nucleic Acids Res 1988; 16(22): 10881-90).

### Results

The AT rich region was next analyzed for the presence of potential motifs or binding sites for recruitment of cellular DNA replication machinery components. The AT region was aligned with an 11 base pair (bp) A region core consensus sequence (ACS) of autonomous replicating sequences (ARS), TAAACATAAAA (SEQ ID No: 4) identified from S. cerevisiae. An 11 bp sequence from the AT region, AAAACTTTAAA (SEQ ID No: 5), was identified that was 75% identical to the ACS (FIG. 5B), providing a likely binding site for recruitment of cellular DNA replication machinery.

### Example 6

### Human ORC2 Binds to the ACS Site of the KSHV Genome

### MATERIALS AND EXPERIMENTAL METHODS

### EMSA

Double-stranded DNA (dsDNA) probes of the sequence indicated in FIG. 5C were prepared from synthetic oligonucleotides (IDT Inc, Coralville, Iowa) by annealing the complementary strand and purifying the dsDNA by 12% native polyacrylamide gel electrophoresis PAGE. Overhangs of probes were filled in with alpha-dCTP using the Klenow fragment of DNA polymerase I. Labeled probes were purified from unincorporated nucleotides with a Nuctrap® probe purification column (Stratagene, La Jolla, Calif.). The EMSA binding reaction mixture contained 25 millimolar (mM) Tris-HCl (pH 7.8), 5 mM MgCl₂, 1 mM ATP, 70 mM KCl, 2 mg/ml BSA, 5 mM DTT, 5% Glycerol, 100 μg/ml of poly(dI/dC), and the indicated labeled probe (100,000 counts per minute [cpm]) per reaction. GST-ORC2 protein was expressed in E. coli and purified using a standard protocol (Verma, S C, Borah, S, et al, 2004. J Virol 78: 10348-10359). GST-ORC2 fusion protein was eluted from Glutathione Sepharose® beads using 10 mM reduced glutathione in 50 mM Tris, pH. 8.0. Protein was concentrated and purified by ultrafiltration using a Centricon 10® (Millipore, USA). 300 nanograms (ng) GST-ORC2 fusion protein and 1 μg α-ORC2 antibody (SantaCruz, Inc. CA), where appropriate, were used per reaction. 200-fold excess cold competitor nucleotide was added 5 min prior to radiolabeled probes. Protein-DNA binding reaction mixture was incubated at 25° C. for 15 min, then reaction mixture was loaded onto a 4.0% polyacrylamide gel containing 0.5×TBE (0.045 M Tris Borate, pH 8.2/1 mM EDTA), which was electrophoresed in 0.5×TBE for 3 h, 150 volts, dried, and exposed to a Phosphorimager® plate (Molecular Dynamics, Inc).

### Chromatin Immunoprecipitation Assay (ChIP)

Cells were crosslinked with 1% formaldehyde by rocking for 10 min at RT, then adding 125 mM glycine. Cells were washed twice with cold PBS, resuspended in cell lysis buffer [5 mM Pipes (KOH), pH 8.0/85 mM KCl/0.5% NP-40] with protease inhibitors, incubated on ice for 10 min, Dounce homogenized, and centrifuged at 5000 rpm, 5 min at 4° C. Nuclei were resuspended in nuclei lysis buffer [50 mM Tris, pH 8.0/10 mM EDTA/1% SDS containing protease inhibitors], incubated for 10 min, and sonicated to obtain an average length of 700 bp. Cell debris was removed by high speed centrifugation for 15 min at 4° C., then the supernatant was removed and diluted 5-fold with ChIP dilution buffer [0.01% SDS/1.0% TritonX-100/1.2 mM EDTA, 16.7 mM Tris, pH 8.1/167 mM NaCl, including protease inhibitors. Samples were pre-cleared with salmon sperm DNA/ProteinA sepharose slurry for 30 min, 4° C., rotating. Supernatants were collected after brief centrifugation. 10% of the total supernatant was saved for use as the input control, and the remainder was divided into 3 fractions and immunoprecipitated with i) control antibody (Sigma, Inc.); ii) Anti-ORC2 (Santa Cruz, Calif.); or iii) anti-MCM3 antibody (Abcam Inc.), followed by addition of salmon sperm DNA/ProteinA/ProteinG slurry. Beads were then washed consecutively with low salt buffer [0.1% SDS/1.0% TritonX-100/2 mM EDTA, 20 mM Tris, pH 8.1/150 mM NaCl); high salt buffer [0.1% SDS/1.0% TritonX-100/2 mM EDTA, 20 mM Tris, pH 8.1/500 mM NaCl]; LiCl wash buffer [0.25M LiCl/1.0% NP40/1% deoxycholate, 1 mM EDTA, 10 mM Tris, pH8.0] and twice in TE (10 mM Tris-HCl, 1 mM EDTA). Complexes were eluted in elution buffer [1% SDS/0.1 molar (M) NaHCO₃] and reverse cross-linked by adding 0.3 M NaCl at 65° C. for 4-5 h. Eluted DNA was precipitated and treated with proteinase K at 45° C., 2 h. Purified DNA was subjected to PCR analysis with the primer set listed in Table 2.

TABLE 2
Primers used for the amplification of
Chromatin Immunoprecipitated (ChIP) DNA:

| | | |
|---|---|---|
| | | SEQ |
| Primer | Sequence | ID No |

| | | |
|---|---|---|
| AT Region | 5′-TGCTAGTATTAATGGTGTTTA-3′ | 6 |
| sense | | |
| AT Region | 5′-TGACGTGCTACCTGTGTA-3′ | 7 |
| antisense | | |
| K5 Region | 5′-TGGCGTCTAAGGACGTAGAAGAGGG-3′ | 8 |
| sense | | |
| K5 Region | 5′-TGCGTGCGCGTGCGGTATAT-3′ | 9 |
| antisense | | |
| G fragment | 5′-TGCTAGTATTAATGGTGTTTA-3′ | 10 |
| sense | | |
| G fragment | 5′-TGACGTGCTACCTGTGTA-3′ | 11 |
| antisense | | |
| E fragment | 5′-ATCCATGCCGCGAGGGTATAGG-3′ | 12 |
| sense | | |
| E fragment | 5′-TATGTTGGTGCCCAGTCGGG-3′ | 13 |
| antisense | | |
| pBS sense | 5′-AGCCCCCGATTTAGAGCT-3′ | 14 |
| pBS antisense | 5′-CAGCCTGAATGGCGAATG-3′ | 15 |
| Puro-ChIP | 5′-CCGCGCAGCAACAGATGGAA-3′ | 21 |
| sense | | |
| Puro-ChIP | 5′-AAGCCGAGCCGCTCGTAGAA-3′ | 22 |
| antisense | | |

### Detection of ORC2 and MCM3 in Immunoprecipitated Chromatin

A fraction of chromatin immunoprecipitated using anti-ORC2 and anti-MCM3 from 293 cells transfected with pBSpuroG, pBSpuroAT, pBSpuroK5 and pBSpuro was resolved on SDS-PAGE and detected using specific antibodies. Western blot shows IP of respective proteins with specific antibodies but not with control IgG antibody (FIG. 8B).

### Results

To determine whether the human replication machinery accumulates in vivo on KSHV AT rich region-containing chromatin, Chromatin Immunoprecipitation (ChIP) analysis was performed on 30 million G1/S-arrested KSHV infected PELs cells (FIG. 5C). BC-3, BCBL1 and JSC-1 cells were cross-linked, and fragmented chromatin was subjected to IP using human α-ORC2 antibody, α-MCM3 antibody and matched control antibody. A fraction of immunoprecipitated chromatin was subjected to western blot analysis for the detection of specific proteins (FIG. 5D). PCR amplification of the AT rich region of the G fragment revealed detection of this fragment with both α-ORC2 and α-MCM3, showing active binding of the replication machinery proteins (FIG. 5F). By contrast, the adjacent regions in the G fragment, K5 ORF did not amplify significant copies of the target DNA, showing that the AT region was responsible for replication protein association. Another region of the Z6 cosmid, E, used as a control for PCR amplification from immunoprecipitated chromatin, did not exhibit detectable amplification (FIG. 5E-F).

Thus, ORCs and MCMs specifically bind to the G region, corroborating the above DpnI sensitivity data and showing that replication of G-region containing plasmids is due to the association of host cellular replication proteins.

To confirm binding of host replication machinery at the AT region, pBSpuroG, pBSpuroAT, pBSpuro K5, and pBSpuro were transfected separately into 293 cells. 48 h post-transfection chromatin prepared from the cells was immunoprecipitated using α-ORC2 and α-MCM3 antibodies, followed by western blot (FIG. 8B). PCR amplification of the region adjacent to the G, AT and K5 inserts on the plasmid in ChIP DNA revealed IP of the AT region, but not the K5 region or vector alone (FIG. 5G).

To confirm that binding of cellular replication machinery proteins is specific to the AT region, chromatin immunoprecipitation (CHIP) using α-ORC2 and α-MCM3 was performed on 293 cells transfected with either pBSpuro gAT or pBSpuro cAT. DNA extracted from the IP revealed amplification of target DNA from plasmid transfected with the AT region, but not from comparable levels of the control AT region plasmid (FIG. 5H). Binding of ORC2 and MCM3 to the AT fragment chromatin was further reproduced in B cell background (DG75) (FIG. 8C).

Thus, cellular replication machinery specifically binds to the AT region of the G fragment.

To test whether the 11 bp sequence described in the previous Example plays a role in initiation of replication, a DNA probe (ACS) was created containing the 11 bp sequence and also 22 bp of upstream sequence. In addition, a mutant probe deleted for the ACS site (Δ858-865) was used to test the specificity of the ORC-DNA interaction. The probes were ³²P-labeled, incubated with bacterially expressed GST-HsORC2 (glutathione S transferase fused to the origin recognition complex 2 protein [HsORC2]), and subjected to electrophoretic mobility shift assay (EMSA). GST-HsORC2 reduced the mobility of the ACS probe, but not the Δ858-865 probe (FIG. 5C; compare lanes 2 and 8), showing that the 11 bp sequence mediated the binding of ORC2 to the AT region. Addition of α-ORC2 antibody supershifted the complex, confirming that ORC2 was present in the DNA-binding complexes (lane 3). ORC2 antibody alone did not show cause supershifting, confirming that the supershift is not due to non-specific binding of the antibody directly to the DNA probes (lane 6). Specific cold competitor, but not non-specific cold competitor, abolished the shift of the probe (lanes 4 and 5, respectively), demonstrating specificity of binding.

These findings show that the 11 bp sequence identified from the AT region mediates binding of the ORC to KSHV genomic DNA.

### Example 7

### Human Origin Recognition Complexes Bind the 11 BP Sequence from the at Region

### Materials and Experimental Methods

### Cell Cycle Arrest

BC-3 and pBSpuroG-transfected 293 cells were treated with thymidine to arrest the cell population at the G1/S boundary. Arrest was confirmed by fluorescence-activated cell sorting (FACS).

### Results

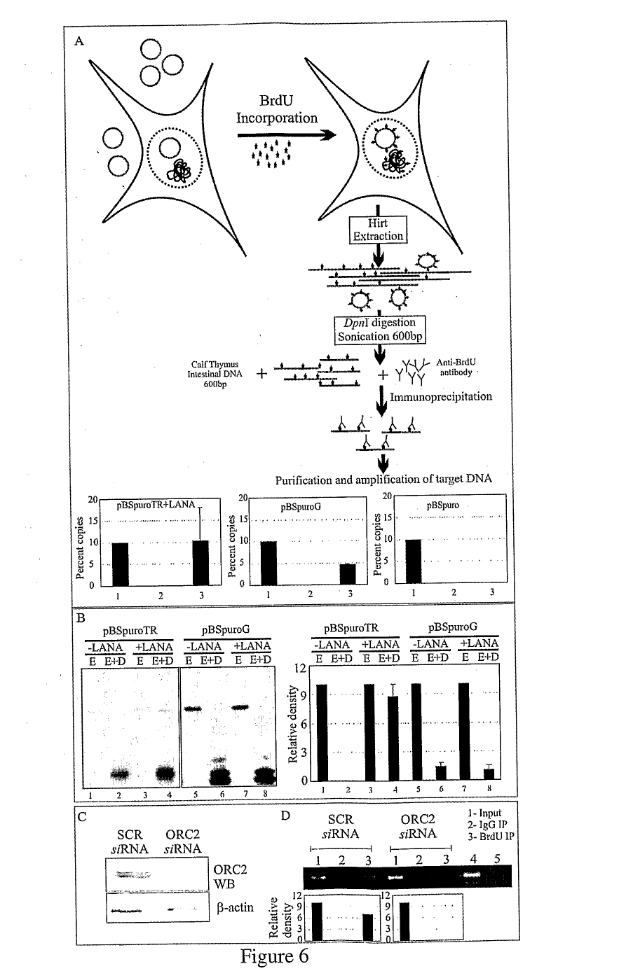

To determine whether the human replication machinery accumulates at the AT region on KSHV chromatin in vivo, Chromatin Immunoprecipitation (ChIP) analysis was performed on G1/S arrested BC-3 and BCBL1 cells. Cells were crosslinked, and chromatin was fragmented and subjected to immunoprecipitation (IP) using human α-ORC2 antibody, α-MCM3 antibody, or a control antibody. A fraction of immunoprecipitated chromatin was subjected to Western blot, which verified the efficacy of the IP (FIG. 2B). Both ORC2 and MCM3 antibodies immunoprecipitated significant amount of chromatin protein (FIG. 6C, lane 1 of each gel). Polymerase chain reaction (PCR) using primers specific for the AT-rich region of the G fragment showed that this fragment was immunoprecipitated by both ORC2 and MCM3 antibodies (FIG. 6B). Thus, the replication machinery binds this sequence in vivo. By contrast, primers specific for adjacent K5 ORF region amplified a much smaller, but detectable, amount of DNA. Primers specific for the E region of the Z6 cosmid did not amplify DNA from the immunoprecipitated chromatin, an result that was expected, based on the short-term assay replication assay described in Example 1 (FIG. 6C; compare lane 3 of each gel).

Chromatin IP was also performed on transiently transfected plasmids containing either the G fragment or the AT-rich region thereof, using α-ORC2 antibody. In both cases, the target DNA was immunoprecipitated, while pBSpuro empty vector (negative control) was not, confirming the previous conclusions that ORC2 associates with the AT region of the G fragment (FIG. 6D).

Thus, human replication machinery accumulates at the AT region on KSHV chromatin in vivo, and corroborate the above findings that binding of ORCs and MCMs is specific to particular DNA elements of the KSHV genome. The small amount of K5 ORF DNA amplified by PCR was likely due to the proximity of the K5 ORF to the AT-rich element, relative to the size of fractionated chromatin DNA; thus, these regions were present together on a subset of the fragments.

### Example 8

### pBSpuroG Actively Incorporates BrdU in Eukaryotic Cells

### Materials and Experimental Methods

### RNA Interference

The siRNA for ORC2 was designed using BLOCK-iT™ RNAi express, siRNA designing tool (Invitrogen, Inc. Carlsbad, Calif.). siRNA ORC2 (5′-AUCCUGAGAUUACGAUAAA-3′; SEQ ID No: 17) and control luciferase (5′-CUUACGCUGAGUACUUCGA-3′; SEQ ID No: 18) was transfected at 100 nM final concentration with Oligofectamine® (Roche Inc. Indianapolis, Ind.). 24 h post-transfection cells were trypsinized and re-plated followed by transfection with 100 nM siRNA and pBSpuroG using Lipofectamine2000® (Invitrogen, Carlsbad, Calif.).

### Anti-BrdU Immunoprecipitation

DNA from BrdU-labeled cells was prepared as described in Example 1. 10% of the DNA was saved to use as input control. FIG. 6A shows the schematic of the BrdU labeling and IP using α-BrdU antibody after sonication. Relative amounts of immunoprecipitated copies were calculated using pBSpuroG as standard in semi quantitative PCR. The remaining BrdU labeled DNA was dissolved in TE to a final volume of 460 μl, followed by addition of 40 μl of 5 mg/ml sheared/denatured salmon sperm DNA. Samples were sonicated to obtain an average length of 700 bp, heat denatured at 95° C., 5 min, and cooled on ice. Samples were adjusted to 10 mM sodium phosphate (pH 7.0), 140 mM NaCl, 0.05% Triton X100, then incubated with 1 μg anti-BrdU antibody (SantaCruz, Inc.) at room temperature with rotation for 1 h, then a 30 min incubation with ProteinA/G. Pellets were washed in 750 μl of 10 mM sodium phosphate buffer, 140 mM NaCl, and 0.05% Triton X100, and resuspended in 200 μl of lysis buffer (50 mM Tris-HCl, pH 8.0, 10 mM EDTA, 0.5% SDS, 0.25 mg/ml proteinase K) and incubated overnight at 37° C. 100 μl additional lysis buffer was added, and samples were incubated at 50° C., 1 h, then phenolized, precipitated, dried and dissolved in 25 μl of sterile water. Prior to IP, samples were precleared with ProteinA/G beads. The resulting pellets had no detectable DNA signals, indicating that the antibodies used for IP were specific for the DNA with incorporated BrdU label.

For PCR amplification 1 μl of the above immunoprecipitated DNA was used in a standard PCR reaction with primers designed to amplify a 420 bp fragment from the puro cDNA using Puro-S; 5′-GCCACATCGAGCGGGTCAC-3′ (SEQ ID No: 23) and Puro-AS; 5′-TCGGCGGTGACGGTGAAG-3′ (SEQ ID No: 24).

### Results

5-Bromo-3-deoxy uridine (BrdU) was used to pulse the replicating 293 cells transfected with pBSpuroG. Amplification of BrdU-immunoprecipitated DNA (FIG. 6A, lane 3) revealed that pBSpuroG was immunoprecipitated by α-BrdU antibodies, but not control IgG antibody (FIG. 6A, lanes 3 and 2, respectively). Thus, the antibody used for IP was specific for BrdU labeled DNA. Since KSHV TR supports replication in the presence of LANA, pBSpuroTR, having a single copy of the TR along with LANA, was used as a control for replication and incorporation of BrdU in a newly synthesized DNA (FIG. 6, pBSpuroTR). Both pBSpuroTR and pBSpuroG incorporated BrdU label. As expected, negative control pBSpuro did not incorporate BrdU (FIG. 6A, pBSpuro).

4-5 fold less replicated copies of pBSpuroG compared to pBSpuroTR in presence of LANA, as revealed by DpnI sensitivity assay (FIG. 6B). Replication of pBSpuroG was unaffected by LANA expression, however, compared to pBSpuroTR which was nearly abolished in the absence of LANA (FIG. 6B).

These results show that BrdU was incorporated into the G region, providing direct evidence for replication of the G region and further confirming previous results. Thus, G fragment-mediated replication is important for the replication of KSHV under conditions wherein the LANA expression is reduced or is highly regulated. Thus, G fragment-mediated replication is critical for complete replication of the KSHV genome.

To further confirm the above results, RNAi was used to deplete ORC2 in 293 cells. ORC2 protein levels detected after 96 h post transfection were significantly reduced in specific siRNA cells but not in scrambled (SCR) siRNA-treated cells (FIG. 6B). pBSpuroG was transfected into the cells and selected for replication along with BrdU to label replicating DNA. Hirt DNA extracted from these cells was digested with DpnI and subjected to IP as in the previous experiment. pBSpuroG was not detectably amplified in the presence of specific siRNA, showing the absence of replication activity in ORC2-depleted cells, while amplification of pBSpuroG in scrambled siRNA-transfected cells was unaffected (FIG. 6C, compare lanes 3 of SCR and ORC2 siRNA). DNA immunoprecipitated with control IgG antibody did not exhibit specific amplification, confirming specificity of IP.

Thus, cellular ORC2 is utilized as a component of the replication machinery of pBSpuroG.

### Example 9

### Analysis of Single Molecule-Replicated DNA Confirms that the G Region Functions as an Origin of Replication

### Materials and Experimental Methods

### IdU Labeling of Replicating DNA and Spreading of Labeled DNA on Glass Slide

HEK293 cells transfected with pBSpuroG and pBSpuro were used for detecting replicated DNA using SMARD. This technique uses 5′-Iodo-2′-deoxyuridine (IdU) and 5′-chloro-2′-deoxyuridine (CldU) to label replicating DNA, then detects incorporated nucleotides by immunofluorescence of individual DNA molecules stretched on glass slides. Exponentially growing cells were pulsed for 4 h with 25 μM of IdU (Sigma-Aldrich) by directly adding to the medium (Norio and Schildkraut, 2004). Cells were collected and washed with cold phosphate-buffered saline by low speed centrifugation. Replicating plasmids were resolved in Gardella gel. The presence of the plasmid band on gel was localized by Southern detection using vector backbone as a probe. The resolved plasmid was extracted from the gel using gel extraction columns.

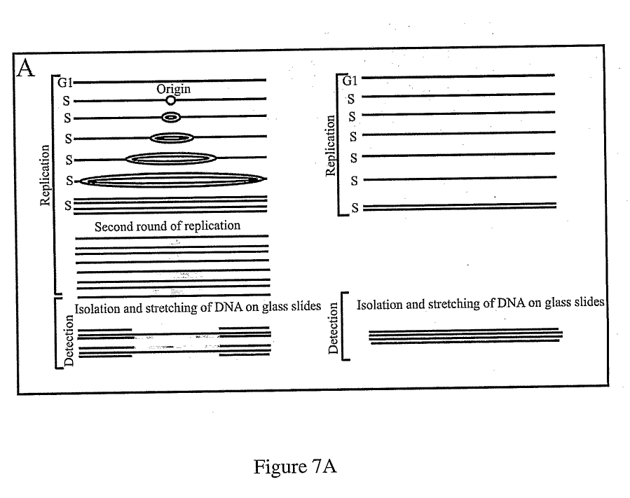

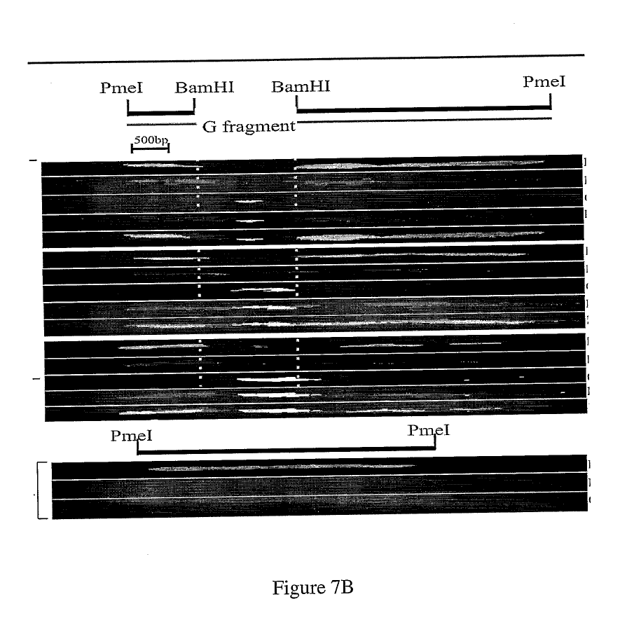

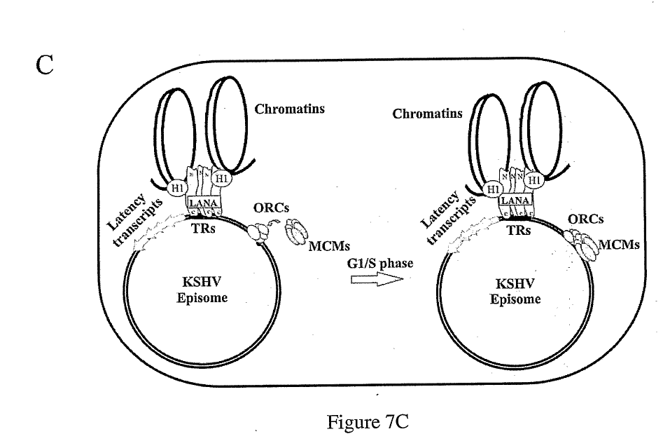

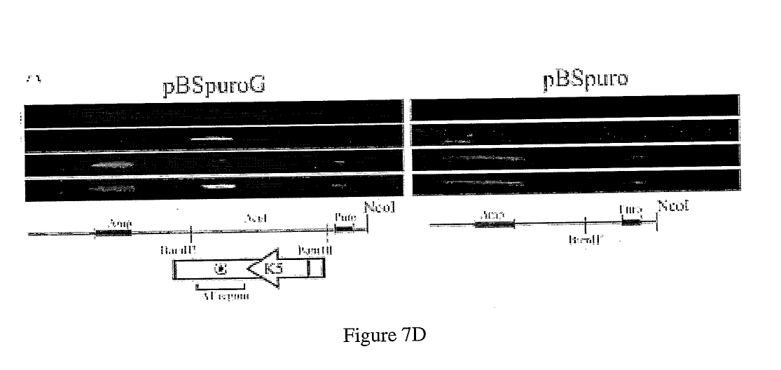

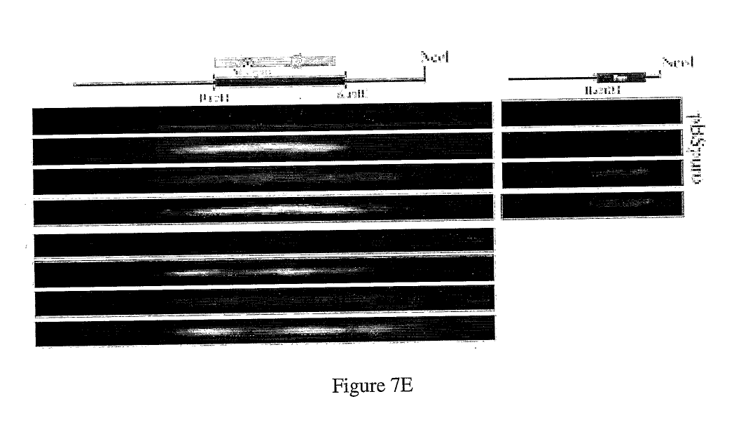

SMARD was performed as described in (Norio, P, and Schildkraut, C L, 2001, Science 294, 2361-2364; and Norio, P, and Schildkraut, C L, 2004, PLoS Biol 2, e152), as described below. 72 h post-transfection cells were labeled with IdU (first label) for 20 h to completely label the entire plasmid length, followed by removal of IdU and a 2 h pulse with CldU (second label). A schematic of double labeling of DNA with halogenated nucleotides in cis-acting replicating DNA elements is depicted in FIG. 7A. DNA was resuspended in printing buffer (3×SSC) and stretched on Super Chip® poly L-lysine-coated slides, using a lifter slip (Erie Scientific, NH), which forms a uniform gap (0.75 mm) between the slide and cover slip, to stretch the DNA uniformly on the slides. Episomal DNA molecules were immobilized on the slides by baking at 85 CC for 1 h. DNA was visualized by hybridizing with biotin labeled probe (NEB). Biotin was detected using Streptavidin conjugated with Alexa Flour 594 (red) (Molecular Probes). Incorporated IdU was detected using monoclonal mouse α-IdU (Becton-Dickinson Inc., Palo Alto, Calif.) as primary antibody and Alexa Fluor 350 (blue) conjugated goat α-mouse (Molecular Probes) as secondary antibody. CldU was detected using rat anti-CldU (Accurate Chemicals, Westbury, N.Y.) and goat anti-rat conjugated with Alexa Flour 488 (green) secondary antibody. DNA molecules were visualized using Olympus BX60 fluorescence microscope and photographs captured using Pixel Fly digital camera (Cooke Inc).

### Results

To directly visualize replication of the G region, the Single Molecule Analysis of Replicated DNA (SMARD) technique was utilized to label 293 cells transiently transfected with pBSpuroG and pBSpuro. Blue signal, indicating the incorporation of IdU, was detected throughout the entire length of pBSpuroG, whereas there was no detectable signal for IdU in pBSpuro empty vector (FIG. 7B). Identification of the transition site due to the incorporation of CldU at the replication initiation was primarily detected in the G region, showing that the G region served as a replication initiation site. Analysis of multiple single DNA molecules showed varying length of CldU incorporation, ranging from a few dots to labeling of the entire G region. 3 representative molecules are depicted in FIG. 7B. Pulsing with the second label was allowed only for a brief period (2-4 h), to predominantly label the replication initiation sites. Transition from IdU to CldU, representing the replication initiation site, was also confirmed on pBSpuroG molecules using different regions of the plasmid as probe (FIG. 7D). pBSpuro did not exhibit detectable incorporation of the halogenated nucleotides, showing specificity of the α-IdU and α-CldU antibodies.

These findings provide further, direct evidence that the G region of the KSHV genome functions as an origin of replication for the KSHV genome, and corroborate the conclusion that the G region serves as a replication initiation site in the KSHV episome (FIG. 7C).

What is claimed is:

1. A method of reducing a replication of a genome of a gammaherpesvirus in a subject, comprising contacting said subject with a composition that inhibits an initiation of DNA replication from a region of a genome of said gammaherpesvirus, wherein the sequence of said region is set forth in SEQ ID No: 2 or a fragment thereof, thereby reducing a replication of a genome of a gammaherpesvirus in a subject.

2. The method of claim 1, wherein said gammaherpesvirus is a rhadinovirus.

3. The method of claim 1, wherein said gammaherpesvirus is a Kaposi's Sarcoma-Associated Herpesvirus (KSHV).

4. The method of claim 3, wherein said replication is a LANA-independent replication.

5. The method of claim 1, wherein said replication is a latent replication.

6. The method of claim 1, wherein said genome is episomal.

7. The method of claim 1, wherein said composition comprises a small molecule inhibitor.

8. The method of claim 1, wherein said composition comprises a peptide nucleic acid (PNA).

9. The method of claim 1, whereby said composition interacts with said region.

10. The method of claim 1, whereby said composition inhibits binding of a DNA replication protein to said region.

11. The method of claim 1, wherein said sequence comprises SEQ ID No: 5.

12. A method of treating a Kaposi's Sarcoma-Associated Herpesvirus (KSHV) infection in a subject, comprising contacting said subject with a composition that inhibits an initiation of DNA replication from a region of a genome of said KSHV, wherein the sequence of said region is set forth in SEQ ID No: 2 or a fragment thereof, thereby treating a KSHV infection in a subject.

13. The method of claim 12, wherein said replication is a LANA-independent replication.

14. The method of claim 12, wherein said replication is a latent replication.

15. The method of claim 12, wherein said genome is episomal.

16. The method of claim 12, wherein said composition comprises a small molecule inhibitor.

17. The method of claim 12, wherein said composition comprises a peptide nucleic acid (PNA).

18. The method of claim 12, whereby said composition interacts with said region.

19. The method of claim 12, whereby said composition inhibits binding of a DNA replication protein to said region.

20. The method of claim 12, wherein said sequence comprises SEQ ID No: 5.

21. A method of treating or reducing an incidence of a Kaposi's Sarcoma-Associated Herpesvirus (KSHV)-associated Kaposi's sarcoma (KS), primary effusion lymphoma (PEL), or multicentric Castleman's disease in a subject, comprising contacting said subject with a composition that inhibits an initiation of DNA replication from a region of a genome of said KSHV, wherein the sequence of said region is set forth in SEQ ID No: 2 or a fragment thereof, thereby treating or reducing an incidence of a KSHV-associated KS, PEL, or multicentric Castleman's disease in a subject.

22. The method of claim 21, wherein said composition comprises a small molecule inhibitor.

23. The method of claim 21, wherein said composition comprises a peptide nucleic acid (PNA).

24. The method of claim 21, whereby said composition interacts with said region.

25. The method of claim 21, whereby said composition inhibits binding of a DNA replication protein to said region.

26. The method of claim 21, wherein said sequence comprises SEQ ID No: 5.

27. An isolated DNA molecule, said isolated DNA molecule comprising (a) a non-Kaposi's Sarcoma-Associated Herpesvirus (KSHV) portion; and (b) a region of a KSHV genome, wherein the sequence of said region is set forth in SEQ ID No: 2 or a fragment thereof, wherein said sequence comprises SEQ ID No: 5.

28. The isolated DNA molecule of claim 27, wherein said isolated DNA molecule is capable of generating self copies in said eukaryotic cell that exhibit long-term persistence in said eukaryotic cell and descendents therefrom.

29. The isolated DNA molecule of claim 27, further comprising a recombinant gene, said recombinant gene encoding a therapeutic protein or therapeutic RNA molecule.

30. A method of delivering a recombinant protein to a subject, comprising administering to said subject the isolated DNA molecule of claim 27, wherein said DNA molecule further comprises a recombinant gene, said recombinant gene encoding said recombinant protein, thereby delivering a recombinant protein to a subject.

31. The method of claim 30, whereby said isolated DNA molecule is capable of generating copies of self that exhibit long-term persistence in said subject, thereby continuing said delivering for at least 3 months following the step of administering.

32. A method of delivering a therapeutic RNA molecule to a subject, comprising administering to said subject the isolated DNA molecule of claim 27, wherein said DNA molecule further comprises a recombinant gene, said recombinant gene encoding said therapeutic RNA molecule, thereby delivering a therapeutic RNA molecule to a subject.

33. The method of claim 32, whereby said isolated DNA molecule is capable of generating copies of self that exhibit long-term persistence in said subject, thereby continuing said delivering for at least 3 months following the step of administering.
